(19) United States
(12) Patent Application Publication
(10) Pub. No.: US 2018/0282397 A1
(43) Pub. Date: Oct. 4, 2018

(54) POLYPEPTIDES THAT BIND ACTIVATED RAS PROTEINS

(71) Applicant: UNIVERSITY OF SOUTHERN CALIFORNIA, Los Angeles, CA (US)

(72) Inventors: Mehmet CETIN, Los Angeles, CA (US); Garrett Gross, Los Angeles, CA (US); Richard Roberts, Los Angeles, CA (US)

(73) Assignee: UNIVERSITY OF SOUTHERN CALIFORNIA, Los Angeles, CA (US)

(21) Appl. No.: 15/844,049

(22) Filed: Dec. 15, 2017

Related U.S. Application Data
(60) Provisional application No. 62/434,911, filed on Dec. 15, 2016.

(51) Int. Cl. C07K 14/82; C07K 14/78; C07K 19/00; C12N 15/79; A61K 38/39

(52) U.S. Cl. CPC C07K 14/82; C07K 14/78; A61P 35/00; C12N 15/79; A61K 38/39; C07K 19/00

(57) ABSTRACT

The invention provides for polypeptides that bind to Ras proteins and methods of using the same, as described herein. For example, the disclosure provides an isolated polypeptide comprising a fibronectin domain, and a first peptide domain at least 90% identical to an amino acid sequence selected from a group consisting of (SEQ ID NO: 6, SEQ ID NO: 7, SEQ ID NO: 8, SEQ ID NO: 9, and SEQ ID NO: 10), and a second peptide domain at least 90% identical to an amino acid sequence selected from a group consisting of (SEQ ID NO: 11, SEQ ID NO: 12, SEQ ID NO: 13, SEQ ID NO: 14, and SEQ ID NO: 15) that specifically binds to an activated Ras protein.

### RELATED APPLICATIONS

This application claims priority under 35 U.S.C. § 119(e) to U.S. Provisional Patent Application No. 62/434,911 filed Dec. 15, 2016, which is incorporated herein by reference.

### GOVERNMENT SUPPORT

This invention was made with government support under grants R01CA170820 and R01GM083898 awarded by the National Institutes of Health. The government has certain rights in the invention.

### FIELD OF THE INVENTION

This disclosure relates to polypeptides that selectively bind to GTP-Ras proteins, and mutants thereof, and methods of using the same.

### BACKGROUND

The Ras family of proteins are a group of closely related monomeric globular proteins of about 189 amino acids (21 kDa molecular mass) that are associated with the plasma membrane. Ras proteins are involved in transmitting signals within cells, and Ras-regulated signal pathways control such processes as actin cytoskeletal integrity, cell proliferation, cell differentiation, cell adhesion, apoptosis, and cell migration. The Ras family of proteins include, for example, K-Ras, H-Ras, N-Ras, Arf1, and Rap1B.

K-Ras and H-Ras have been identified as mediators of malignant characteristics, and oncogenic mutations in these proteins are commonly observed. Missense mutations in human Ras were some of the first oncogenes discovered, with multiple research groups reporting that the H-Ras G12V mutation may transform certain cell lines. Ras overactivation is associated with 71% of pancreatic cancers, 35% of colon cancers, and 19% of lung cancers.

Ras may bind either Guanadine Diphosphate (GDP) or Guanadine Triphosphate (GTP) to act as a molecular “switch”. Ras—when bound with GDP (GDP-RAS)—is in the resting or “off” position and the protein is inactive. In response to certain external cellular stimuli, Ras may exchange a bound GDP molecule for a GTP molecule (GTP-RAS). The binding of GTP causes Ras to enter an “on” state, enabling the activated GTP-RAS to interact with, and activate downstream target proteins. The Ras protein itself has a very low intrinsic ability to hydrolyze GTP back to GDP, thus, once on, Ras generally remains in the “on” state. Ras inactivation—that is, a return to the inactive state—requires various extrinsic proteins termed GTPase-activating proteins (GAPs) that interact with Ras, and greatly accelerate the conversion of GTP to GDP. Any Ras mutation that affects the ability to interact with GAP, or to convert GTP back to GDP may result in a prolonged activation and, consequently, prolong downstream signaling, that may lead to a cancerous phenotype.

Structurally, Ras proteins contain a G-domain responsible for the enzymatic activity, that is, the guanine nucleotide binding and exchange reaction, as well as the GTPase hydrolysis reaction. Ras also contains a C-terminal extension, known as the CAAX box, which may be post-translationally modified, and is responsible for targeting the protein to the membrane. The G-domain is approximately 20 kDa in size and contains a phosphate binding loop (P-loop). The P-loop includes the nucleotide pocket, defined in part by the amino acid residues essential for nucleotide binding and hydrolysis (Glycine 12, Threonine 26 and Lysine 16). The G-domain further includes the “Switch I” (residues 30-40) and “Switch II” (residues 60-76) regions, each of which are dynamic parts of the protein that are termed the “spring-loaded” mechanism because of their ability to switch between the resting and active state. Importantly, Ras activity includes the formation of hydrogen bonds between Threonine-35, Glycine-60, and the γ-phosphate of the GTP molecule, that maintain the Switch 1 and Switch 2 regions, respectively, in the active conformation. After hydrolysis of GTP and release of phosphate, the switch domains may revert into the inactive GDP bound conformation.

Accordingly, molecules that may regulate oncogenic Ras function may be of immense value as targeted therapeutics. However, despite decades of extensive research, currently there are no therapies directly targeting Ras. As a result, Ras continues to be considered an “undruggable target.”

Recent attempts to inhibit Ras signaling have focused on blocking the Switch I and Switch II regions to prevent Ras interaction with downstream effectors. Antibodies, cyclic peptides and compounds that covalently modify mutant Ras, have previously been generated, and many of these molecules can disrupt Ras signaling. However, while these molecules may modulate Ras activity under certain conditions, each has drawbacks that limit their potential use, including a lack of state specificity, that is, active versus inactive state, off-target effects, large size, and/or loss of efficacy within a cell. The present invention addresses these needs.

### SUMMARY OF THE INVENTION

In one aspect, the disclosure relates to polypeptides that bind to a Ras protein. The polypeptides may comprise at least a scaffold domain and one or more binding domains. In preferred embodiments of the peptides of the invention, the scaffold domain is derived from fibronectin, and more preferably, from the 10th domain of fibronectin. One certain embodiment of the invention includes the 10FnIII or e10FnIII fibronectin domain.

The one or more binding domains may be configured to bind to a specific protein. In preferred embodiments, the one more binding domains are configured to bind to at least a portion of a Ras protein, a Ras homolog, or a Ras protein mutant protein. While embodiments of a polypeptide of the invention may bind to the Ras protein in any structural conformation, preferentially the polypeptide binds to a Ras protein in the active state, that is, when Ras is actively bound with GTP or a similar analog thereof.

In certain embodiments of the invention, a first binding domain is selected from SEQ ID NO: 6-10 and a second binding domain is selected from SEQ ID NO: 11-15. In other preferred embodiments, the polypeptide has an amino acid sequence of any one of SEQ ID No: 1-4.

Certain embodiments of the invention may bind to a mutant Ras protein having a G12V point mutation, or both a G12V and Y32R point mutation. Further embodiments of the invention may bind to the Ras switch I or switch II domain.

In a further aspect, the disclosure relates to certain embodiments of methods of treating cancer in a subject using an inventive polypeptide. The method may include administering to the subject a therapeutically effective amount of a polypeptide as detailed herein.

Another aspect of the disclosure provides methods of detecting cancer in a sample from subject using an inventive polypeptide. The method may include contacting the sample with a polypeptide as detailed herein. Certain preferred embodiments may further include a detectable label covalently linked to the polypeptide such as GFP or enhanced GFP.

Another aspect of the disclosure provides a polynucleotide encoding the polypeptide as detailed herein. Another aspect of the disclosure provides an expression vector comprising the polynucleotide.

The disclosure provides for other aspects and embodiments that will be apparent in view of the following detailed description and accompanying figures.

### BRIEF DESCRIPTION OF THE DRAWINGS

The preferred embodiments of the invention will be described in conjunction with the appended drawings provided to illustrate and not to the limit the invention, where like designations denote like elements, and in which:

FIG. 1. Illustrates an mRNA display selection against K-Ras(G12V)-GTPγS using the e10FnIII scaffold. (a) e10FnIII scaffold and library design (PDB ID:1FNF). The BC loop (SEQ ID NO: 82) and FC loop are shown. Backbone mutations relative to wild-type 10FnIII are shown in yellow. In order to direct the library to recognize active Ras, variants of e10FnIII were constructed where the BC loop was a doped sequence from a previous Ras ligand (iDab#6). BC loop doping was ˜40% wild-type at each residue. The FG loop was a naïve random sequence. (b) In vitro selection for K-Ras(G12V)-GTPγS. Pool binding was measured for matrix without target (neutravidin or streptavidin agarose) or for immobilized K-Ras exchanged with GTPγS or GDP. No binding is observed in matrix without target, while preferential binding to K-Ras(GTPγS) over K-Ras(GDP) is observed in Round 6. (c) Individual clones were screened for function inside the cells using a cellular co-localization assay. Scale bar represents 5 μm. (d) Screening resulted in the identification of a Ras-specific clone termed RasIn1 (SEQ ID NO: 1).

FIG. 2. Illustrates the binding characteristics of RasIn1 in vitro. (a) RasIn1 preferentially binds active (GTP) over inactive (GDP) forms of H-Ras(G12V) (p=0.007) and preferentially binds unblocked active H-Ras(G12V) as active H-Ras(G12V) blocked with the c-Raf Raf-kinase RBD domain (Raf-RBD; p=0.002). (b) Binding of RasIn1 to K-Ras is disrupted by the Y32R mutation in the Switch I region (p=0.03). Error bars indicate the standard deviation of the mean.

FIG. 3. Illustrates the binding of RasIn1 to different Ras homologs. (a) Radiolabeled RasIn1 shows excellent binding to different versions of Ras (wild-type K- or H-Ras, and G12V mutant K- or H-Ras), showing that the recognition of the active Ras state is robust. (b) Binding specificity of RasIn1 against homologous members of the Ras superfamily. RasIn1 binds specifically to active K-Ras, but less to homologous Ras family members Rap1B(G12V) (p=0.01) or Arf1 (0.02) (percentage of sequence identity to the K-Ras G domain is shown in parentheses). Error bars indicate the standard deviation of the mean.

FIG. 4. Illustrates colocalization of RasIn1 with various Ras forms in COS-7 cells. RasIn1-EGFP was co-transfected with Golgi-targeting sequence-streptavidin (GTS-SA) (a-c), GTS-SA-wild-type H-Ras (d-f), or GTS-SA-H-Ras(G12V) (g-i). Cells were fixed and stained for EGFP and streptavidin. Colocalization is indicated in the merged image. Images are representative of at least 15 samples. Scale bar represents 5 μm.

FIG. 5 illustrates a mutational analysis of RasIn1 reveals functionally important residues. (a) In vitro pull-down efficiency of RasIn1 point mutants. Radiolabeled point mutants of RasIn1 were tested for binding to immobilized K-Ras-GTP. The percentage of radioactive counts bound to beads is shown. S24, R72, and R77 are critical positions for binding. (b) BC (SEQ ID NO: 6) and FG loop (SEQ ID NO: 11) mutations observed in the top 20 single point mutants of RasIn1 in high-throughput sequencing of pool 6 (Table 1). Residue numbers are shown above the sequence.

FIG. 6. Illustrates a selection of RasIn2. (a) Enrichment of Ras-binding sequences through affinity maturation. Pool 5 shows high levels of binding to active H-Ras(G12V). (b) In vitro pull-down efficiency of high abundance sequences from pool 5 in comparison with RasIn1. (c) Sequence comparison of high abundance sequences from pool 5 showing the BC and FC loops of RasIn1 (SEQ ID NO:6; SEQ ID NO: 11), Clone 1 (SEQ ID NO: 8; SEQ ID NO: 13), Clone 2 (SEQ ID NO: 9; SEQ ID NO: 14), RasIn2 (SEQ ID NO: 7; SEQ ID NO: 12), and clone 4 (SEQ ID NO: 10; SEQ ID NO: 15).

FIG. 7. Illustrates the characterization of RasIn2 binding. (a) RasIn2 shows binding to different versions of active Ras (wild-type K- or H-Ras, and G12V mutant K- or H-Ras), showing that the recognition of the active Ras state is robust. (b) RasIn2 preferentially binds active (GTP) over inactive (GDP) forms of H-Ras(G12V) (p=0.003) and preferentially binds unblocked active H-Ras(G12V) over active H-Ras(G12V) blocked with the c-Raf Raf-kinase RBD domain (Raf-RBD; p=0.002). (c) Binding specificity of RasIn2 against homologous members of the Ras superfamily. RasIn1 binds specifically to active wild-type H-Ras, but less to homologous Ras family members Rap1B(G12V) (p=0.0001) or Arf1 (p=0.0002). Percentage of sequence identity to the H-Ras G domain is shown in parentheses. (d) Binding of RasIn2 to K-Ras is disrupted by the Y32R mutation in the Switch I region (p=0.002). (e) Dissociation constants of RasIn1 and RasIn2 for H-Ras(G12V)-GTP, determined by SPR). Error bars indicate the standard deviation of the mean.

FIG. 8. illustrates an ELISA assay indicates that RasIn2 has higher affinity for H-Ras(G12V)-GppNHp than Raf1-RBD. (a) Schematic of the ELISA assay. Biotinylated H-Ras(G12V) was captured on the ELISA plate via streptavidin-biotin link. FLAG-tagged RasIn2 or Raf-RBD was then incubated with the ELISA plate, followed by an anti-FLAG antibody, and then a secondary antibody conjugated to horseradish peroxidase (HRP). The plate was then washed and incubated with tetramethylbenzidine (TMB). (b) The OD450 of the ELISA readout plotted over different concentrations of ligand (RasIn2 or Raf-RBD). RasIn2 shows ˜10-fold higher specific signal level than Raf-RBD.

FIG. 9. Illustrates RasIn2 colocalizes with various Ras forms in COS-7 cells. COS cells were co-transfected with RasIn2-GFP and GTS-SA-H-Ras(G12V) (a-c), GTS-SA-wt H-Ras (d-f), or SA-GTS (no target; g-i). Cells were fixed and stained for EGFP and streptavidin. Colocalization is visible in the merged image. RasIn2 colocalizes with both active (GTP) and inactive (GDP) H-Ras. Images are representative of at least 15 samples. Scale bar represents 5 μm.

### BRIEF DESCRIPTION OF THE TABLE

Table 1 on page 32 below shows RasIn1 single point mutants found in high throughput sequence data. The 20 highest copy number mutants are included in the analysis. Rank within the pool, BC and FG loop sequences, and copy number are shown for each clone. Mutant residues are shown underlined.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Cancer” refers to a group of diseases involving abnormal cell growth with the potential to invade or spread to other parts of the body. Cancers may include, but are not limited to, breast cancer, colorectal cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. In some embodiments, the cancer is breast cancer.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. Biometrics 2000, 56, 337-44, the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be a subject, or a sample therefrom, whose disease state is known. The subject, or sample therefrom, may be healthy, diseased, diseased prior to treatment, diseased during treatment, or diseased after treatment, or a combination thereof.

The term “expression vector” indicates a plasmid, a virus or another medium, known in the art, into which a nucleic acid sequence for encoding a desired protein can be inserted or introduced.

The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Intrabody” as used herein refers to a polypeptide that binds a target. An intrabody may be antibody-like in that they may be specific for a target, and they may include immunoglobulin-like folds with loops that are structurally similar to antibody CDRH1 and CDRH3 regions.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids.

“Recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

“Reporter,” “reporter group,” “label,” and “detectable label” are used interchangeably herein. The reporter is capable of generating a detectable signal. The label can produce a signal that is detectable by visual or instrumental means. A variety of reporter groups can be used, differing in the physical nature of signal transduction (e.g., fluorescence, electrochemical, nuclear magnetic resonance (NMR), and electron paramagnetic resonance (EPR)) and in the chemical nature of the reporter group. Various reporters include signal-producing substances, such as chromagens, fluorescent compounds, chemiluminescent compounds, radioactive compounds, and the like. In some embodiments, the reporter comprises a radiolabel. Reporters may include moieties that produce light, e.g., acridinium compounds, and moieties that produce fluorescence, e.g., fluorescein. In some embodiments, the signal from the reporter is a fluorescent signal. The reporter may comprise a fluorophore. Examples of fluorophores include, but are not limited to, acrylodan (6-acryloyl-2-dimethylaminonaphthalene), badan (6-bromo-acetyl-2-dimethylamino-naphthalene), rhodamine, naphthalene, danzyl aziridine, 4-[N-[(2-iodoacetoxy)ethyl]-N-methylamino]-7-nitrobenz-2-oxa-1,3-diazole ester (IANBDE), 44N-[(2-iodoacetoxy)ethyl]-N-methylamino-7-nitrobenz-2-oxa-1,3-diazole (IANBDA), fluorescein, dipyrrometheneboron difluoride (BODIPY), 4-nitrobenzo[c][1,2,5]oxadiazole (NBD), Alexa fluorescent dyes, and derivatives thereof. Fluorescein derivatives may include, for example, 5-fluorescein, 6-carboxyfluorescein, 3′6-carboxyfluorescein, 5(6)-carboxyfluorescein, 6-hexachlorofluorescein, 6-tetrachlorofluorescein, fluorescein, and isothiocyanate.

“Sample” or “test sample” as used herein can mean any sample in which the presence and/or level of a target is to be detected or determined. The target may include Ras or a member of the Ras family. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchioalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “specificity” as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having a disease when they do not in fact have disease. Hence, a method that has both sensitivity and specificity equaling one, or 100%, is preferred.

By “specifically binds,” it is generally meant that a polypeptide binds to a target when it binds to that target more readily than it would bind to a random, unrelated target. The target may include Ras or a member of the Ras family.

“Subject” as used herein can mean a mammal that wants or is in need of the herein described compositions. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

“Target” as used herein can refer to an entity that a polypeptide binds. A target may include, for example, a small molecule, a protein, a polypeptide, a polynucleotide, a carbohydrate, or a combination thereof. The target may include Ras or a member of the Ras family.

“Treatment” or “treating,” when referring to protection of a subject from a disease, means preventing, suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease. The disease may be cancer.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequence substantially identical thereto. A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

Described herein are novel polypeptides that are selective for a Ras protein such as H-Ras, N-Ras, and K-Ras. The rationale for targeting Ras is that the chronic signaling of K- and H-Ras, by mutation or upstream activation, occurs in more than 50% of human tumors. Therefore, embodiments of the polypeptides disclosed herein selectively bind K-Ras and H-Ras proteins relative to other Ras protein family members, as well as the active forms of K-Ras and H-Ras to detect and modulate activated Ras signaling.

Preferably, polypeptides of the invention include the following properties: (1) selectivity for the GTP-bound state of Ras with little or no binding to related homologs and (2) functionality in the cytosolic environment. The result of the present disclosure is the development a group of intrabodies that bind to mutant and wild-type K- and H-Ras in a GTP-state-dependent fashion. The polypeptides of the invention may also be selective for Ras G12V mutants, which are mutant Ras proteins constitutively locked in the active, GTP-bound state, and are found in numerous cancers. The disclosed polypeptides may also bind to other forms of Ras including, for example, GTP-bound K-Ras, GTP-bound H-Ras, GDP-bound K-Ras, nucleotide-free K-Ras, GDP-bound H-Ras, and nucleotide-free H-Ras.

Preferred embodiments of the polypeptides include a fibronectin domain as a scaffold. Various methods may be used to generating fibronectins with high affinity to specific targets. The fibronectin scaffold (e.g., 10FnIII) may be based on the 10th fibronectin type III domain of human fibronectin and has an immunoglobulin-like fold with loops that are structurally similar to antibody CDRH1 and CDRH3 regions. The 10FnIII scaffold is small (10 kDa), lacks disulfides, can be expressed in Escherichia coli, and is an alternative to antibodies for generating affinity reagents and intrabodies. Advantageously, one preferred embodiment of the fibronectin domain—termed e10FnIII—improves solubility and expression in E. coli and in the reticulocyte lysate translation system.

Certain embodiments of Ras binding proteins—termed “RasIns”—preferably include e10FnIII-based and disulfide-free intrabodies against activated (GTP-bound) Ras proteins. These fibronectin-based proteins selectively bind the active state of Ras and are specific for Ras over homologous proteins. At the same time, these fibronectin-based proteins bind both H- and K-Ras-GTP selectively for mutants. Furthermore, these proteins are functional when expressed inside a cell, and may show comparable binding affinities for Ras-GTP with the Raf-kinase RBD domain (Raf-RBD) (the canonical binding partner of Ras).

Preferred embodiments of a polypeptide that binds a Ras protein comprise a fibronectin domain, and one or more binding domains configured to specifically binds Ras. The fibronectin domain—such as from human fibronectin—may include the 10th domain of fibronectin (SEQ ID NO: 17). In some embodiments, the fibronectin domain comprises 10FnIII (SEQ ID NO:18) or e10FnIII (SEQ ID NO: 19).

Certain embodiments of the polypeptides of the invention include one or more binding domains such as a BC loop domain, or an FG loop domain. Preferably, an embodiment of a polypeptide includes both a BC loop domain and an FG loop domain. Certain embodiments of the invention include a BC loop is at least 80%, at least 85%, at least 90%, or at least 95% identical to any one of SEQ ID. NO: 6-10, and the FG loop is at least 80%, at least 85%, at least 90%, or at least 95% identical to any one of SEQ ID NO: 11-15.

One preferred embodiment of the invention includes a polypeptide comprising an e10FnIII domain, a BC loop domain, and an FC loop domain. In certain embodiments, the polypeptide may be a polypeptide of any one of SEQ ID NO: 1-4.

In some embodiments, the polypeptide selectively binds a G12V mutant K-Ras (SEQ ID NO: 26) relative to wild-type K-Ras (SEQ ID NO:24). In some embodiments, the polypeptide selectively binds a G12V mutant H-Ras (SEQ ID NO: 27) relative to wild-type H-Ras (SEQ ID NO:25.

In some embodiments, the polypeptide selectively binds wild-type H-Ras and a G12V mutant H-Ras relative to wild-type K-Ras and a G12V mutant K-Ras.

In some embodiments, the polypeptide selectively binds Ras protein relative to other Ras family members selected from Arf1 and Rap1B.

In some embodiments, the polypeptide binds the Switch 1 (SEQ ID NO: 16; SEQ ID NO: 22; SEQ ID NO: 23) domain of a Ras protein.

In some embodiments, the K_{D} of the polypeptide for the Ras protein may be less than 5 μM, less than 2.5 μM, less than 200 nM, or less than 150 nM.

In some embodiments, the polypeptide selectively binds GTP-bound K-Ras or GTP-bound H-Ras relative to GDP-bound K-Ras, nucleotide-free K-Ras, GDP-bound H-Ras, or nucleotide-free H-Ras. In some embodiments, the polypeptide selectively binds GTP-bound H-Ras relative to GDP-bound H-Ras or nucleotide-free H-Ras.

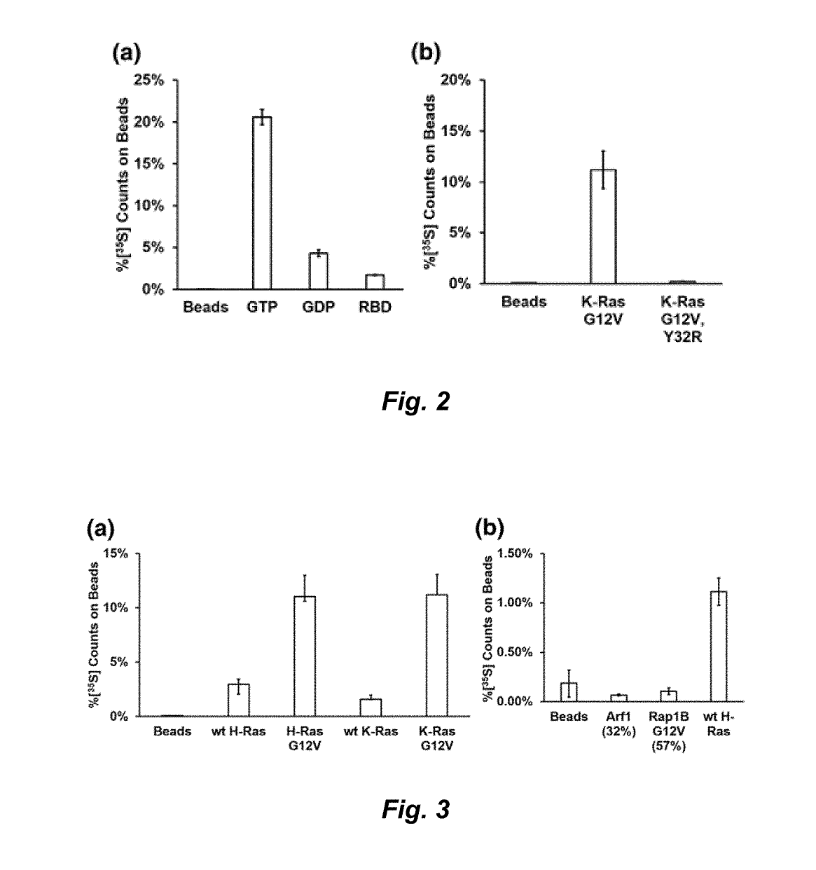

In more specific embodiments of the invention, both RasIn1 (SEQ ID NO: 1) and RasIn2 (SEQ ID NO: 2) satisfy these binding specificity criteria. RasIn1 has a K_{D} of 2.1 μM, a reasonable outcome from a primary selection with 17 random/randomized residues. It is notable that this affinity is sufficient to provide very good co-localization with the intended target in the COS cells, even though this affinity is less than the downstream partner Raf-RBD for the active state of Ras (KD=80 nM). RasIn1 also has excellent nucleotide, mutant, and homolog selectivity, showing little or no binding to Ras-GDP (FIG. 2a) or Rap1B-GTP (FIG. 3b). This result is remarkable because Ras (SEQ ID NO: 25 or 26) and Rap1B (SEQ ID NO: 20) are 57% sequence identical, and the Switch I sequences of Ras (SEQ ID NO: 16 YPDTIED) and Rap1B (SEQ ID NO: 22 YDPTIED) differ by only 2 amino acids. Mutation and competition analysis of the RasIn1 indicates that the Ras binding overlaps the known Ras binding site for Raf-RBD, although the precise sequence interactions must be different—the recognition sequences overall show basic character (R/K rich) and have no sequence homology.

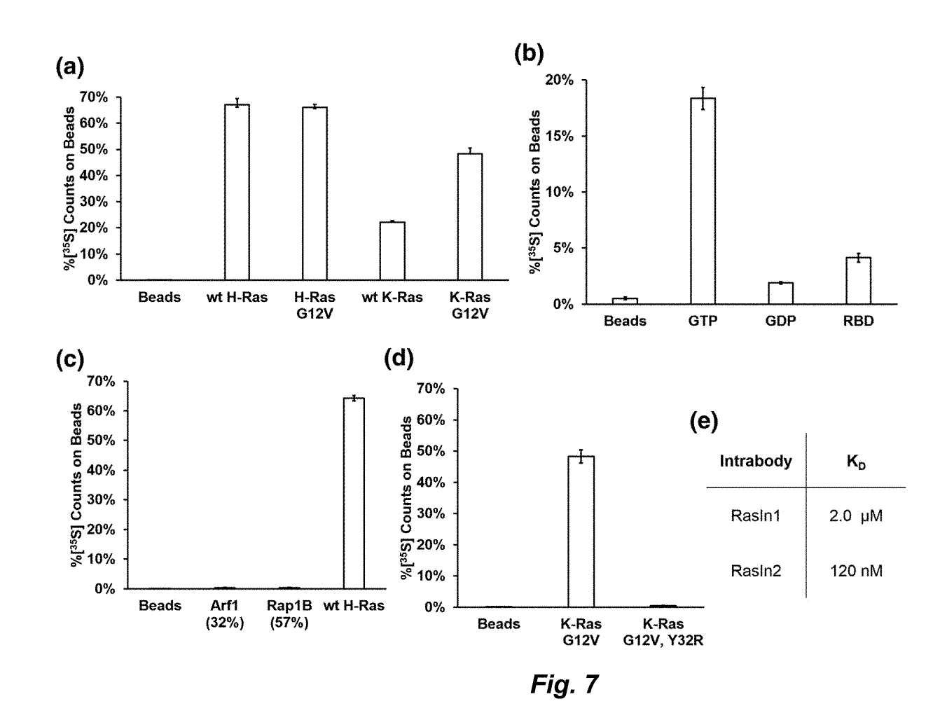

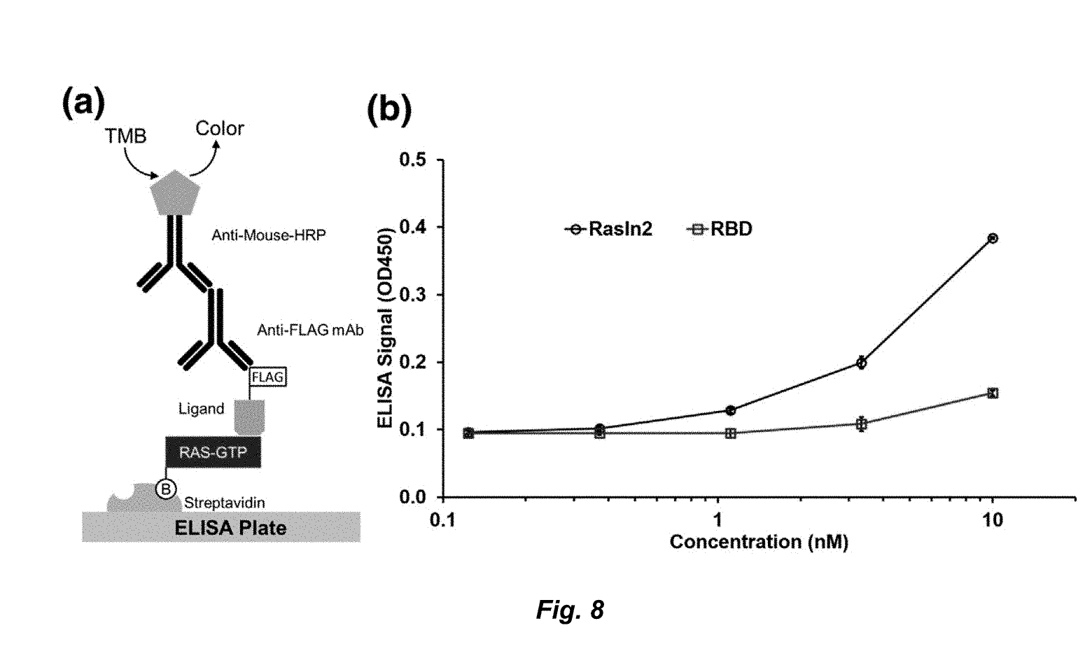

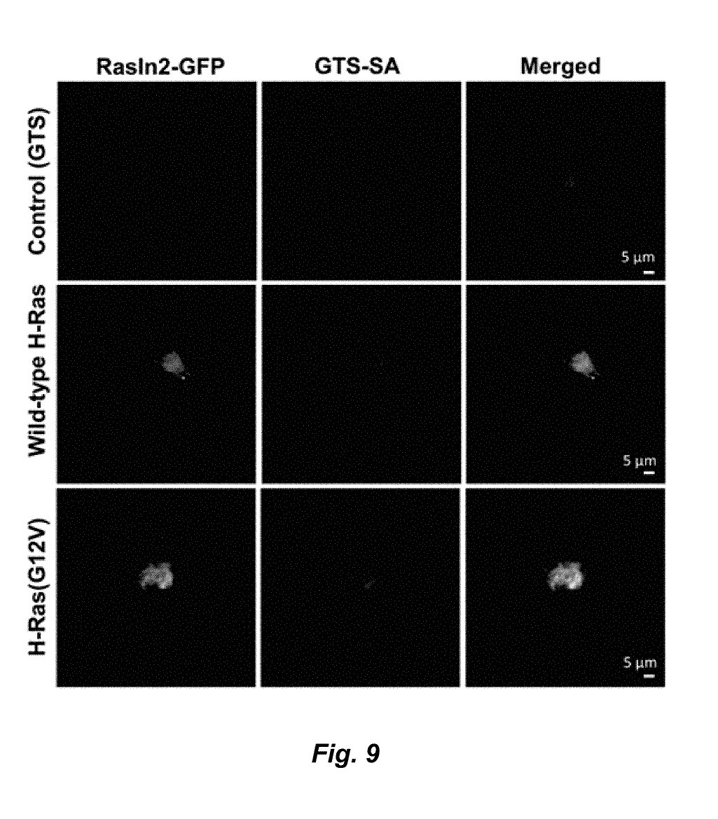

Affinity maturation allows for the enrichment of protein-protein interactions. For example, affinity maturation of RasIn1 resulted in the high affinity binder RasIn2, with a K_{D} of 120 nM and a 20-fold improvement gained through sequence optimization of the recognition site. Interestingly, there are 8 conserved positions in the FG loop, even though the loop sequences are mutagenized at >65% per position, such that the 10-residue loop is expected to have between 3 and 4 conserved positions. (see Olson et al., mRNA display selection of a high-affinity, modification-specific phosphor-I kappa B alpha-binding fibronectin, ACS Chem. Biol. 3 (2008) 480-485; Xiao et al. Antibody-mimetic ligand selected by mRNA display targets DC-SIGN for dendridic cell-directed antigen delivery, ACS Chem Biol. 8 (2013) 967-977). RasIn2 showed markedly improved pulldown with Ras compared to RasIn1 (FIG. 6b), competes with Raf-RBD (FIG. 7b), shows broad recognition biochemically of K-Ras(G12V)-GTP, wild-type K-Ras-GTP, H-Ras(G12V)-GTP, and wild-type H-Ras-GTP (FIG. 7a), outperforms RBD in an ELISA assay (FIG. 8), and also shows excellent co-localization with H-Ras(G12V) and wild-type H-Ras inside COS cells (FIG. 9). The fact that RasIn1 and RasIn2 show similar intensity in the COS cell assay is likely a result of the high transient expression levels of both the binder (RasIn) and the target (Ras) in the COS cells.

Overall, the two proteins—RasIn1 and RasIn2—demonstrate that it is possible to use mRNA display to engineer individual, Ras-directed reagents with broad specificity for both the mutant and active state of K- and H-Ras. Furthermore, the selectivity of these binders indicates it may be possible to develop similar reagents for other G-proteins and study other G-protein-mediated pathways.

Embodiments of the invention may also be used to study various cellular effects on Ras activity. For example, the mutant selectivity of RasIn1 and the high affinity of RasIn2 indicated that these proteins may be able to modulate or block downstream signaling via Ras kinase activation, a long-sought goal in cancer therapeutics. RasIn1 or RasIn2 also could be developed into active Ras biosensors for in vitro or in vivo studies, histology, or cancer diagnostics. Their small size, high affinity, lack of disulfide bonds, state selectivity, Ras specificity, and ability to be transfected into cells lend RasIn1 and RasIn2 to a wide range of possible future uses that often restrict the application of antibodies or non-genetically encoded molecules.

In a preferred embodiment, the polypeptides—or intrabody—comprises another functional domain such as an enzyme, e.g., a protease, that may lead to the proteolysis of all or parts the polypeptide. In yet another embodiment, the polypeptide of the invention comprises a targeting signal that is capable of retargeting the polypeptide to another cellular locale. For, example, such a locale may be cytoplasmic, nuclear, lysosomal, plasma membrane-associated, endoplasmic reticulum-associated, peroxisomal, or proteasomal. In addition, the intrabodies or binding molecules of the invention may encompass any art recognized targeting signal for altering the cellular location of a heterologous polypeptide such as those described in any one of U.S. Pat. Nos. 5,132,405; 5,091,513; 5,084,398; 5,525,491; and 5,851,829, or International Patent Application WO99/14353, which are incorporated herein by reference in their entirety.

Embodiments of the polypeptides disclosed herein may also comprise a cell-penetrating peptide. As used herein, “cell-penetrating polypeptide” or “CPP” refers to a polypeptide which may facilitate the cellular uptake of molecules. A polypeptide-CPP fusion protein of the present invention may include one or more detectable labels. The polypeptides may be partially labeled or completely labeled throughout. The CPP may also include a signal sequence that may be used to signal the secretion of the CPP.

Embodiments of the invention also may include more than one binding site or copies of a single binding site, and a number of other functional regions. For example, multivalent intrabodies are included within the scope of the invention and such intrabodies may have an affinity for one or more epitopes found within the selected polypeptide. Moreover, in a preferred embodiment, the multivalent intrabody may have one or more affinities for an epitope found within a normal peptide in addition to one or more affinities to an epitope found in an altered polypeptide. In another embodiment, the intrabody may have affinity for one or more epitopes found within the altered polypeptide, for example, a polypeptide associated with disease (e.g. a mutant Ras protein). In another embodiment, the multivalent intrabody may have the ability to selectively bind an altered polypeptide that may have a different half-life than that of the normal polypeptide, accumulate at a different rate or in a different cellular space (or be secreted), assume an altered conformation, aggregate (with itself or other polypeptides), form undesired interactions with other polypeptides, cause altered cell growth or cell death, and/or cause a disorder or disease.

The ability to design the intrabody of the invention depends on the ability to determine the sequence of the amino acids of the polypeptide of interest, or the DNA encoding them. Accordingly, the present invention further provides polynucleotides encoding the polypeptides detailed herein. The processes for manipulating, amplifying, and recombining DNA which encode amino acid sequences of a polypeptide are generally well known in the art. Methods of identifying and isolating genes encoding polypeptides of interest are described herein, and for example, in U.S. Pat. Nos. 5,132,405; 5,091,513; 5,084,398; 5,525,491; 5,851,829, and international patent application WO99/14353 which are hereby incorporated by reference.

After obtaining the polynucleotide encoding the polypeptides detailed herein, the sequence may be inserted into a vector. To obtain expression of a polypeptide, one may clone the polynucleotide encoding the polypeptide into an expression vector that contains a promoter to direct transcription, a transcription/translation terminator, and if for a nucleic acid encoding a protein, a ribosome binding site for translational initiation.

As used herein, vector (or plasmid) refers to discrete elements that are used to introduce heterologous DNA into cells for expression thereof. Selection and use of such vehicles are well within the skill of the artisan. Many vectors are available, and selection of appropriate vector will depend on the intended use of the vector, the size of the nucleic acid to be inserted into the vector, and the host cell to be transformed with the vector. Each vector contains various components depending on its function and the host cell for which it is compatible. The vector components generally include, but are not limited to, one or more of the following: an origin of replication, one or more marker genes, an enhancer element, a promoter, a transcription termination sequence and a signal sequence.

Moreover, nucleic acids encoding the polypeptides and/or targets according to the invention may be incorporated into cloning vectors, for general manipulation and nucleic acid amplification purposes.

Both expression and cloning vectors generally contain nucleic acid sequence that enable the vector to replicate in one or more selected host cells. Typically, in cloning vectors, this sequence is one that enables the vector to replicate independently of the host chromosomal DNA, and includes origins of replication or autonomously replicating sequences. Such sequences are well known for a variety of bacteria, yeast and viruses.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media. As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for pro to trophy in an auxotrophic yeast mutant, for example the URA3, LEU2, LYS2, TRPI, ADE2 or HIS3 gene. Since the replication of vectors is conveniently done in E. coli, an E. coli genetic marker and an E. coli origin of replication are advantageously included. These can be obtained from E. coli plasmids, such as pBR322, BluescriptC) vector or a pUC plasmid, e.g. pUC18 or pUC19, which contain both an E. coli replication origin and an E. coli genetic marker conferring resistance to antibiotics, such as ampicillin.

Suitable selectable markers for mammalian cells are those that enable the identification of cells expressing the desired nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked nucleic acid. Amplification is the process by which genes in greater demand for the production of a protein critical for growth, together with closely associated genes which may encode a desired protein, are reiterated in tandem within the chromosomes of recombinant cells. Increased quantities of desired protein are usually synthesized from thus amplified DNA. Expression and cloning vectors usually contain a promoter that is recognized by the host organism and is operably linked to the desired nucleic acid. Such a promoter may be inducible or constitutive. The promoters are operably linked to the nucleic acid by removing the promoter from the source DNA and inserting the isolated promoter sequence into the vector. Both the native promoter sequence and many heterologous promoters may be used to direct amplification and/or expression of nucleic acid encoding the immunoglobulin or target molecule.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Promoters suitable for use with prokaryotic hosts-include, for example, the p-lactamase and. lactose promoter systems, alkaline phosphatase, the tryptophan (trp) promoter system and hybrid promoters such as the tac promoter. Preferred expression vectors include bacterial expression vectors which comprise a promoter of a bacteriophage such as phage φ_{X} or T7 that is capable of functioning in the bacteria.

Suitable promoter sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a-factor or a promoter derived from a gene encoding a glycolytic enzyme such as the promoter of the enolase, glyceraldehyde-3phosphate dehydrogenase (GAP), 3-phospho glycerate kinase (PGK), hexokinase, pyruvate decarboxylase, phosphofructokinase, glucose-6-phosphate isomerase, 3phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (TBP) gene may be used. Furthermore, it is possible to use hybrid promoters comprising upstream activation sequences (UAS) of one yeast gene and downstream promoter elements including a functional TATA box of another yeast gene, for example a hybrid promoter including the UAS (s) of the yeast. PH05 gene and downstream promoter elements including a functional TATA box of the yeast GAP gene (PH05-GAP hybrid promoter).

A suitable constitutive PH05 promoter is e.g. a shortened acid phosphatase PH05 promoter devoid of the upstream regulatory elements (UAS) such as the PH05 (-173) promoter element starting at nucleotide-173 and ending at nucleoli de-9 of the PH05 gene. Gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from promoters normally associated with immunoglobulin sequences. Transcription of a nucleic acid by higher eukaryotes may be increased by inserting an enhancer sequence into the vector. Eukaryotic expression vectors will also contain sequences necessary for the termination of transcription and for stabilizing the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding the immunoglobulin or the target.

Particularly useful for practicing the present invention are expression vectors that provide for the transient expression of nucleic acids in mammalian cells. Transient expression usually involves the use of an expression vector that can replicate efficiently in a host cell, such that the host cell accumulates many copies of the expression vector, and, in turn, synthesizes high levels of the desired gene product. Construction of vectors according to the invention may employ conventional ligation techniques. Isolated plasmids or DNA fragments are cleaved, tailored, and re-ligated in the form desired to generate the plasmids required. If desired, analysis to confirm correct sequences in the constructed plasmids is performed in a known fashion.

Suitable methods for constructing expression vectors, preparing in vitro transcripts, introducing DNA into host cells, and performing analyses for assessing gene product expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridization, using an appropriately labelled probe which may be based on a sequence provided herein. Those skilled in the art will readily envisage how these methods may be modified, if desired. Immunoglobulins and/or targets may be directly introduced to the cell by microinjection, or delivery using vesicles such as liposomes which are capable of fusing with the cell membrane. Viral fusogenic peptides are advantageously used to promote membrane fusion and delivery to the cytoplasm of the cell.

The polypeptide also may be expressed recombinantly in a host cell according to one of skill in the art. The polypeptide may be purified by any means known to one of skill in the art. For example, the polypeptide may be purified using chromatography, such as liquid chromatography, size exclusion chromatography, or affinity chromatography, or a combination thereof.

The polypeptides as detailed herein may be formulated into a composition in accordance with standard techniques well known to those skilled in the pharmaceutical art. The composition may be prepared for administration to a subject in dosages and by techniques well known to those skilled in the medical arts taking into consideration such factors as the age, sex, weight, and condition of the particular subject, and the route of administration.

The polypeptide may be administered prophylactically or therapeutically. In prophylactic administration, the polypeptide can be administered in an amount sufficient to induce a response. In therapeutic applications, the polypeptides can be administered to a subject in need thereof in an amount sufficient to elicit a therapeutic effect. An amount adequate to accomplish this is defined as “therapeutically effective dose.” Amounts effective for this use will depend on, e.g., the particular composition of the polypeptide regimen administered, the manner of administration, the stage and severity of the disease, the general state of health of the patient, and the judgment of the prescribing physician.

In certain embodiments, the therapeutic and prophylactic compositions may comprise a purified form of an inventive polypeptide that binds to a Ras protein. The disclosed polypeptides may be formulated into a pharmaceutical composition, where the polypeptide is present in amounts ranging from about 0.01% (w/w) to about 100% (w/w), from about 0.1% (w/w) to about 80% (w/w), from about 1% (w/w) to about 70% (w/w), from about 10% (w/w) to about 60% (w/w), or from about 0.1% (w/w) to about 20% (w/w).

One skilled in the art will appreciate that a variety of suitable methods of administering a polypeptide to a subject or host, e.g., patient, in need thereof, are available, and, although more than one route can be used to administer a particular composition, a particular route may provide a more immediate and more effective reaction than another route. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the polypeptide is administered intravenously, intraarterially, or intraperitoneally to the subject.

Pharmaceutically acceptable excipients are also well known to those who are skilled in the art, and are readily available. The choice of excipient will be determined in part by the particular compound, as well as by the particular method used to administer the composition. Accordingly, there are a wide variety of suitable formulations of the polypeptide compositions. The following methods and excipients are merely exemplary and are in no way limiting.

Formulations suitable for oral administration may consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; (d) suitable emulsions and (e) hydrogels. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are known in the art.

Polypeptide formulations may be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, patches, sprays or foams.

Suppository formulations are also provided by mixing with a variety of bases such as emulsifying bases or water-soluble bases. Formulations suitable for vaginal administration may be presented as pessaries, tampons, creams, gels, pastes, foams.

Optionally, the pharmaceutical composition may contain other pharmaceutically acceptable components, such as buffers, surfactants, antioxidants, viscosity modifying agents, preservatives and the like. Each of these components is well-known in the art. See, e.g., U.S. Pat. No. 5,985,310, the disclosure of which is herein incorporated by reference.

The present compositions can be administered alone, or may be administered in combination with radiation, surgery, or another therapy (e.g., a chemotherapeutic agent, an angiogenesis inhibitor), to treat a disease such as cancer. Treatments may be sequential, with a disclosed polypeptide being administered before or after the administration of other therapy. For example, a polypeptide of the invention, or composition thereof, may be used to sensitize a cancer patient to radiation or chemotherapy. Alternatively, treatments may be administered concurrently.

Non-limiting examples of chemotherapeutic agents include, but are not limited to, a DNA alkylating agent, a topoisomerase inhibitor, an endoplasmic reticulum stress inducing agent, a platinum agent, an antimetabolite, a vincalkaloid, a taxane, an epothilone, an enzyme inhibitor, a receptor antagonist, a tyrosine kinase inhibitor, a boron radiosensitizer (e.g., velcade), and combinations thereof.

Exemplary of enzyme inhibitors include, but are not limited to farnesyltransferase inhibitors (Tipifarnib); CDK inhibitor (Alvocidib, Seliciclib); proteasome inhibitor (Bortezomib); phosphodiesterase inhibitor (Anagrelide; rolipram); IMP dehydrogenase inhibitor (Tiazofurine); and lipoxygenase inhibitor (Masoprocol). Examples of receptor antagonists include, but are not limited to ERA (Atrasentan); retinoid X receptor (Bexarotene); and a sex steroid (Testolactone).

Exemplary tyrosine kinase inhibitors include, but are not limited to inhibitors to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib). A chemical equivalent of lapatinib is a small molecule or agent that is a tyrosine kinase inhibitor (TKI) or alternatively a HER-1 inhibitor or a HER-2 inhibitor. Several TKIs have been found to have effective antitumor activity and have been approved or are in clinical trials. Examples of such include, but are not limited to, Zactima (ZD6474), Iressa (gefitinib), imatinib mesylate (STI571; Gleevec), erlotinib (OSI-1774; Tarceva), canertinib (CI 1033), semaxinib (SU5416), vatalanib (PTK787/ZK222584), sorafenib (BAY 43-9006), sutent (SUI 1248) and lefltmomide (SU101).

Polypeptides of the invention may be co-administered with antiviral agents, anti-inflammatory agents or antibiotics. The agents may be administered concurrently or sequentially. The present agents can be administered before, during or after the administration of the other therapy.

Embodiments of the polypeptide also may be used as a means for detecting Ras, or a Ras mutant protein. As used herein, the term “detect” or determine the presence of refers to the qualitative measurement of undetectable, low, normal, or high concentrations of one or more polypeptides bound to Ras. Detection may include in vitro, ex vivo, or in vivo detection. Detection may include detecting the presence of one or more Ras proteins, Ras mutant proteins, or portions thereof versus the absence of the one or more Ras proteins. Detection may also include quantification of the level of one or more Ras proteins, Ras mutant proteins, or portions thereof.

The term “quantify” or “quantification” may be used interchangeably, and may refer to a process of determining the quantity or abundance of a substance (e.g., polypeptide or Ras protein), whether relative or absolute. Any suitable method of detection falls within the general scope of the present disclosure. In some embodiments, the polypeptide comprises a reporter attached thereto for detection. In some embodiments, the polypeptide is labeled with a reporter.

In preferred embodiments the detectable label may be a fluorophore. The fluorophore may be a fluorescent protein such as GFP or eGFP. GFP or eGFP may be synthesized together with the intrabody polypeptide by expression therewith as a fusion protein, according to methods well known in the art. For example, a transcription unit may be constructed as an in-frame fusion of the desired GFP and the intrabody polypeptide, and inserted into a vector as described above, using conventional PCR cloning and ligation techniques.

In some embodiments, detection of a polypeptide bound to Ras may be determined by methods including but not limited to, band intensity on a Western blot, flow cytometry, radiolabel imaging, cell binding assays, activity assays, SPR, immunoassay, or by various other methods known in the art.

Further provided herein are methods of treating cancer in a subject. The method may include administering an effective amount of the polypeptide or composition comprising a polypeptide described herein to the subject.

Treatment using the a polypeptide of the invention, or composition thereof, may have one or more of the following effects on cancer cells or the subject: cell death; decreased cell proliferation; decreased numbers of cells; inhibition of cell growth; apoptosis; necrosis; mitotic catastrophe; cell cycle arrest; decreased cell size; decreased cell division; decreased cell survival; decreased cell metabolism; markers of cell damage or cytotoxicity; indirect indicators of cell damage or cytotoxicity such as tumor shrinkage; improved survival of a subject; or disappearance of markers associated with undesirable, unwanted, or aberrant cell proliferation.

Cancers that may be treated by the present agents include, but are not limited to, lung cancer; ear, nose and throat cancer; nervous system cancers; brain cancer; leukemia; colon cancer; melanoma; pancreatic cancer; mammary cancer; prostate cancer; breast cancer; hematopoietic cancer; ovarian cancer; basal cell carcinoma; biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; liver cancer; lymphoma including Hodgkin's and Non-Hodgkin's lymphoma; myeloma; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Further provided herein are methods detecting cancer in a sample from subject comprising detecting the level of activated Ras protein in a sample from the subject and comparing the level of activated Ras protein to the level of Ras protein in a control sample, where an increased level of activated Ras protein identifies the subject as at risk of having cancer or as having cancer.

In some embodiments, the detection of the polypeptide bound to Ras or a mutant Ras protein may indicate cancer in the subject. For example, the presence of a polypeptide bound to a Ras protein having a G12V point mutation, or a G12V and Y32R point mutations (SEQ ID NO: 28; SEQ ID NO: 29) may indicate a certain cancer. In some embodiments, detected levels of the Ras that are less or more than a control sample may indicate cancer.

In further embodiments of the invention, downstream signaling molecules may be assayed to determine the presence or absence of certain cancers by, for example, comparing the activity of certain downstream signaling molecules of a known reference sample to the patient sample suspected of having a cancer. An increase in the amount of the downstream signaling protein, or the increase or decrease in the amount of downstream signaling protein activity may indicate the presence or absence of a cancer.

In certain embodiments, the one or more downstream effector proteins may include members of a mitogen activated protein kinase (MAPK) signaling pathway, including, but not limited to, the extracellular signal regulated mitogen-activated protein kinase (ERK-MAPK) signaling pathway. The MAPK signaling pathway is a main component in several steps of tumorigenesis including cancer cell proliferation, migration, invasion and survival. Any component of the MAPK signaling pathway or the ERK pathway may be the effector proteins, including, but not limited to, RAF, MEK, MAPK (ERK), or combinations thereof. Any isoform of any component the MAPK pathway may be the effector proteins, including, but not limited to, BRAF, CRAF, ARAF; MEK1, MEK2, MKK3, MKK4, MKKS, MKK6, or MKK7; ERK1, ERK2, p38, JNK, ERKS, or combinations thereof.

Other non-limiting examples of downstream Ras effectors include, Abl, Aurora-A, Aurora-B, Aurora-C, ATK, bcr-Abl, Blk, Brk, Btk, c-Kit, c-Met, c-Src, CDK1, CDK2, CDK4, CDK6, cRafl, CSFIR, CSK, EGFR, ErbB2, ErbB3, ErbB4, ERK, Fak, fes, FGFR1, FGFR2, FGFR3, FGFR4, FGFR5, Fgr, FLK-4, Flt-1, Fms, Fps, Frk, Fyn, Hck, IGF-1R, INS-R, Jak, KDR, Lck, Lyn, MEK, p38, PDGFR, PIK, PKC, PYK2, Ros, Tie1, Tie2, Trk, Yes, Zap70, and combinations thereof. (Magnuson et al., Seminars in Cancer Biology, 5:247-252 (1994)).

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Protein Expression and Purification.

pCDNA 3.1 vector (Invitrogen)-encoding Ras family genes were obtained from Missouri S&T cDNA Resource Center. K-Ras(G12V) and H-Ras (G12V) (1-166) with a 5′ avitag and His tag were cloned into pET16b or pET24a vector. Proteins were expressed in E. coli BL21 (DE3) containing plasmid pBirAcm (Avidity) that overexpresses the birA biotin ligase for in vivo biotinylation. Cells were grown at 30° C. for 4-5 h after induction with 1 mM IPTG at an OD of 0.4, and cells were pelleted and frozen at −80° C. Cells were lysed with French Press or BPER (Thermo Scientific), and the lysate was cleared by centrifugation and applied to a Ni(II)-NTA column (Qiagen) pre-equilibrated with binding buffer [50 mM Tris-HCl or Hepes (pH 7.5), 100 mM NaCl, 0.05% (vol/vol) Tween-20, 1 mM MgCl2, 10 μM GDP, 1 mM DTT, and 20 mM imidazole]. The column was washed with wash buffer (binding buffer with 500 mM NaCl and 40 mM imidazole), and proteins were eluted with a linear gradient of wash buffer and elution buffer (binding buffer with 20 mM NaCl and 400 mM imidazole). The molecular weight and purity of the protein were confirmed by denaturing SDS-PAGE. Purified protein was buffer exchanged into selection buffer [50 mM Tris-HCl (pH 7.5), 150 mM NaCl, 0.05% (vol/vol) Tween-20, 5 mM MgCl2, and 1 mM DTT] using Amicon Ultra spin columns, supplemented with 2 mM GDP and 10% (vol/vol) glycerol, and then frozen in liquid nitrogen. The activity of Ras proteins was confirmed by an in vitro pull-down assay with Raf-RBD.

DNA-encoding c-Raf-RBD (c-Raf residues 51-131) was obtained from Integrated DNA Technologies. c-Raf-RBD, RasIn1, and RasIn2 were cloned into the pAO9 vector, which fuses each protein to a C-terminal maltose binding protein. Proteins were expressed in E. coli BL21 (DE3) and purified on a Ni(II)-NTA column as described above. RasIn1 and RasIn2 were subjected to a secondary purification step on an amylose column (New England Biolabs). Proteins were stored at −80° C. in storage buffer [50 mM Tris-HCl (pH 7.5), 150 mM NaCl, 0.05% (vol/vol) Tween-20, and 1 mM DTT].

Rap1B(G12V) (1-166), RhoA (1-181), and Arf1 (18-179) were cloned in pDW363 and expressed in E. coli BL21 (DE3) pBirAcm for 5 h after induction with 0.5 mM IPTG. Cells were pelleted and frozen at −80° C. Pellets were lysed with BPER, lysate was cleared by centrifugation, and the biotinylated proteins immobilized on neutravidin agarose beads (Thermo Scientific). Rap1B(G12V) functionality was validated with a radioactive pull-down of its canonical binding partner, Ral-GDS.

K-Ras(G12V, Y32R) mutants were generated by PCR-based site-directed mutagenesis using pDW363 K-Ras(G12V) as template. Proteins were expressed and purified as described for Ras family member proteins.

Nucleotide Exchange.

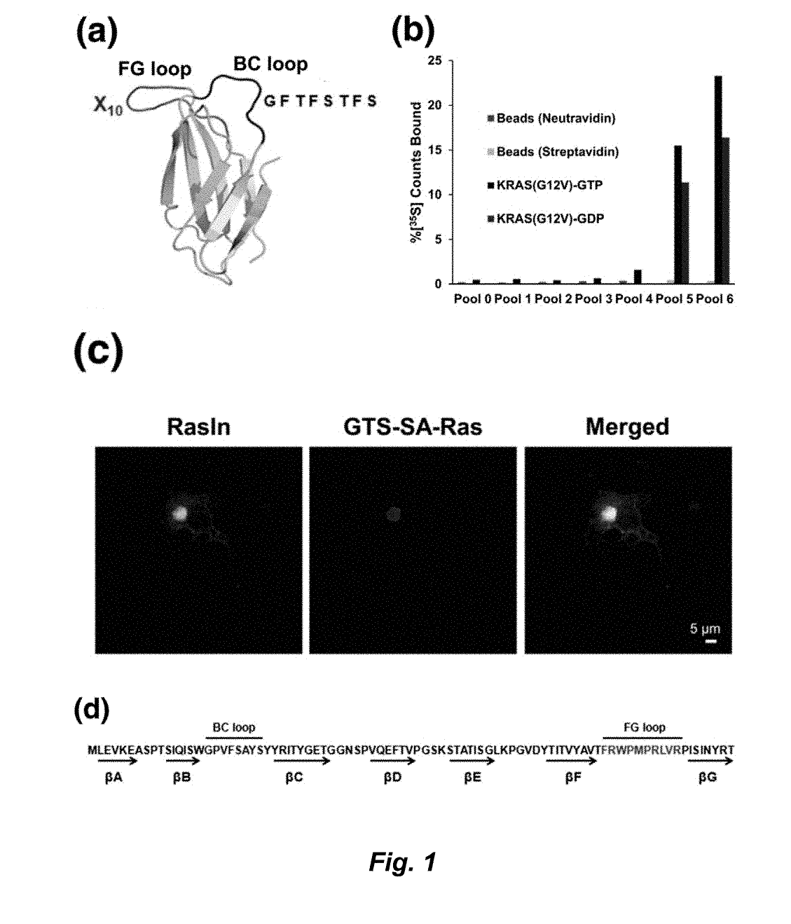

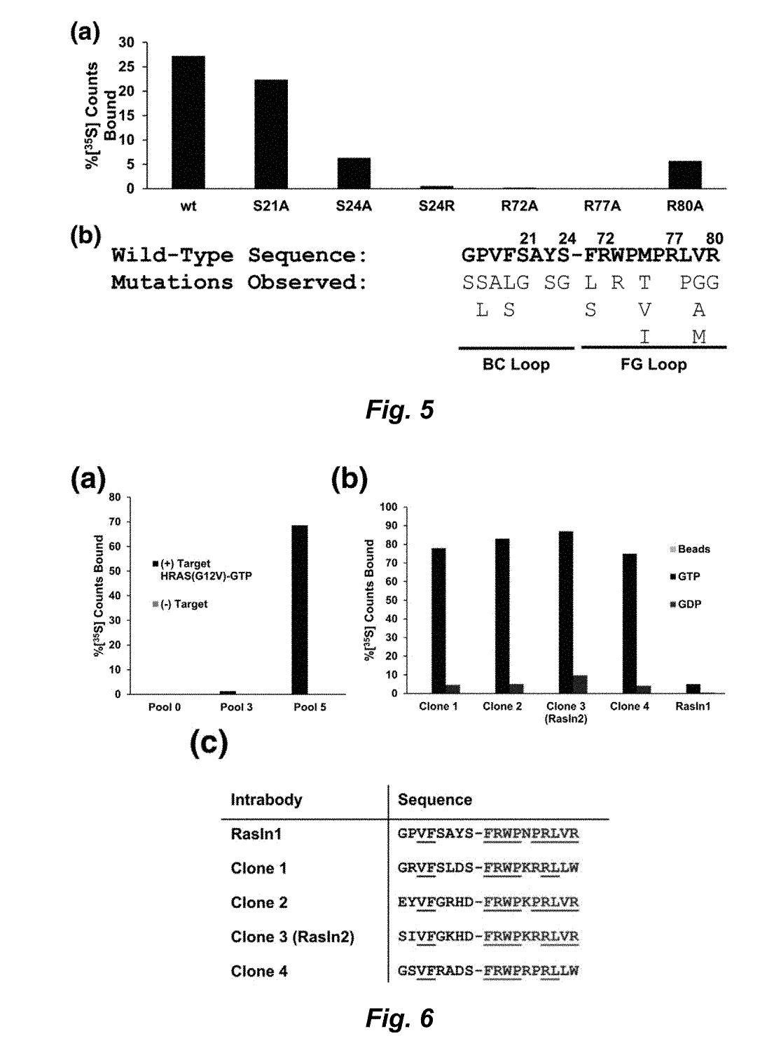

For data in FIG. 1, FIG. 5, and FIG. 6, Ras proteins were immobilized on neutravidin agarose (Thermo Fisher) or Dynabeads® M-280 streptavidin magnetic beads (Life Technologies) and washed three times with exchange buffer [50 mM Tris-HCl (pH 7.5), 5 mM EDTA, 150 mM NaCl, and 1 mM nucleotide]. Beads were incubated at 30° C. for 30 min, transferred on ice, and washed three times with ice-cold selection buffer to stop exchange.

For the binding assays in FIG. 2, FIG. 3, and FIG. 7, nucleotide exchange was facilitated by incubating the Ras beads in selection buffer (20 mM Hepes, 200 mM NaCl, 200 μM nucleotide, and 0.05% TWEEN) for 3 h.

Library Preparation.

Libraries were built as described (Olson et al., Design, expression, and stability of a diverse protein library based on the human fibronectin type III domain, Protein Sci. 16 (2007) 476-484), except that the Arg-24 position in e10FnIII was randomized to create a random region totaling eight residues in the BC loop. Oligos encoding the random regions were synthesized by the Yale Keck Oligonucleotide Synthesis facility.

mRNA Display Selection.

The FG loop contained 10 random residues (X10; where X=all 20 natural amino acids, encoded by the NNS codon), and the BC loop of the library was a doped sequence of the CDRH1 loop of iDab#6, an antibody with state-specific affinity for Ras, with approximately 40% frequency at each residue for the initial selection. For the first round, the library was translated in a total volume of 2.5 mL, purified, and reverse transcribed. K-Ras(G12V) was immobilized on neutravidin agarose beads and exchanged with GTPγS as described above. The library was then added to immobilized K-Ras(G12V)GTPγS in selection buffer [50 mM Tris-HCl (pH 7.5), 150 mM NaCl, 0.05% (vol/vol) Tween-20, 5 mM MgCl2, 1 mM DTT, and 10 μM GTPγS] at 4° C. Following the first round, a FLAG purification step was included after translation to purify mRNA-fibronectin fusions away from unfused mRNA, and the target binding step was performed at 25° C. At round 3, streptavidin-ultralink beads (Thermo Fisher) were used to prevent the enrichment of neutravidin agarose binding sequences. At round 5, a 15-fold molar excess of K-Ras(G12V)-GDP was added to the selection buffer to enhance selective binding for active Ras.

Affinity Maturation.

A new library was synthesized containing BC and FG loops that were doped on the RasIn1 sequence (BC=GPVFSAYS (SEQ ID NO: 6) and FG=FRWPMPRLVR (SEQ ID NO:11) for the affinity maturation selection. The frequency of wild-type RasIn1 in the starting pool was estimated to be approximately 1.8 in 108 sequences. For the first round of selection, 200 μL of library was translated. H-Ras(G12V) was immobilized on 40 μL of Dynabeads® M-280 streptavidin magnetic beads and was exchanged with GppNHp. Immobilized H-Ras(G12V) and purified library were incubated as above. Binding for the first round was performed at 4° C., rounds 2 and 3 were performed at 25° C., and rounds 4 and 5 were performed at 37° C. to increase binding stringency.

Radioactive In Vitro Pull-Down Assays.

Ras proteins were immobilized and exchanged with nucleotide as described above. Individual ligands or selection pools were radiolabeled by translation in vitro in rabbit reticulocyte lysate in the presence of [35S] Met. After oligo-dT or FLAG purification, radiolabeled ligands were added to Ras beads, incubated for the indicated duration, and washed three times with selection buffer. The percentage of total radioactive counts remaining on the beads was determined by scintillation counting.

Cell-Based Co-Localization Assay and Immunocytochemistry.

The cell-based co-localization assay and immuno-cytochemistry were done as previously described (Gross et al., Recombinant probes for visualizing endogenous synaptic proteins in living neurons, Neuron 78 (2013) 971-985.). Briefly, COS-7 cells (ATCC) were grown to a confluency of ˜40-60% on poly-D-lysine-coated 22×22 mm glass coverslips in Dulbecco's modified eagle's medium (ATCC) supplemented with 10% fetal bovine serum in 5% CO2. pGW mammalian expression plasmids assembled with Golgi-targeted Ras-Streptavidin or 10FnIII-EGFP fusion genes inserted into the MCS were then co-transfected into cells using Effectene transfection reagent with transfection rates of around 10-30% (Qiagen). Following 24 h of plasmid expression, COS cells were washed once with PBS, fixed with 4% paraformaldehyde for 5 min, washed three times with PBS, blocked for 30 min in blocking buffer [1% bovine serum albumin, 5% normal goat serum, 0.1% (vol/vol) Triton X-100 in PBS], and stained for 1 h with chicken anti-GFP antibody (Ayes), diluted 1:1000 in blocking buffer. After incubation with primary antibody, cells were washed three times with PBS and stained for 30 min with anti-chicken secondary antibody conjugated to Alexa Fluor 488 (Invitrogen), 1:1000, and Biotin-Rhodamine conjugate (VWR), 1:000, in blocking buffer. Cells were then washed three times with PBS and mounted on 75×25 mm glass microscope slides in Fluoromount-G (Electron Microscopy Sciences) for imaging. All steps were carried out at room temperature. Images of cells were taken with a 60×water objective at 1.0 zoom on an Olympus IX81 inverted microscope equipped with a GFP/mCherry filter cube (Chroma Technology), an X-cite exacte mercury lamp (Excelitas Technologies), an EM-CCD digital camera (Hamamatsu), and Metamorph software (Molecular Devices). Cells were scored for the co-localization of Rhodamine and Alexa Fluor 488 fluorescence. In each sample, at least 15 cells were imaged, and co-localization was observed either every time or never.

High-Throughput Sequencing.

Selection pools were sequenced at the USC Epigenome Center and the USC Genome & Cytometry Core using MiSeg™ and HiSeg™ and Systems (Illumina). Data analysis was done using computer scripts developed in-house.

SPR Measurements.

Measurements were done in USC NanoBiophysics Core Facility on a Biacore T100 instrument (Biacore). H-Ras(G12V) was immobilized on streptavidin sensor chips at the indicated surface density. Ras protein was exchanged with GppNHp on a chip where indicated by injecting SPR exchange buffer [50 mM Tris-HCl (pH 7.5), 1 mM EDTA, and 0.5 mM GppNHp] at 35 μL/min for 10 min. A concentration series of maltose binding protein-intrabody fusion was injected at a flow rate of 100 μL/min at 25° C. in SPR run buffer [50 mM Tris-HCl (pH 7.5), 150 mM NaCl, 0.01% (vol/vol) Tween-20, 5 mM MgCl2, and 10 μM GppNHp]. Data were analyzed by Biacore T100 Evaluation Software.

ELISA Assay.

The polystyrene plate was incubated with 50 μL of 30 nM streptavidin in 1×PBS per well overnight at 4° C. The plate was washed with 1×TBS with 0.1% (vol/vol) Tween-20, filled completely with 5% BSA (wt/vol) in 1×PBS, incubated for 3 h, and washed. Biotin-labeled H-Ras(G12V)-GppNHp was diluted in sample buffer [1×TBS, 5 mM MgCl2, 1 mM DTT, 20 μM GppNHp, 0.1% (vol/vol) Tween-20, and 1 mg/mL BSA]. 100 uL of 30 nM biotin-labeled H-Ras(G12V)-GppNHp was added to each well, and it was incubated for 90 min. After the incubation, 100 μL of exchange buffer (50 mM Tris, 5 mM EDTA, 1 mM DTT, 600 μM GppNHp, and 1 μM biotin) was added to each well and incubated for 30 min, and the plate was then washed in wash buffer [1×TBS, 5 mM MgCl2, 50 μM GTP, and 0.1% (vol/vol Tween-20]. RasIn2 protein and RBD were diluted serially in sample buffer and added to the plate. The plate was incubated for 1 h and washed. 100 μL of 20 nM Anti-FLAG antibody (Sigma) was added to the plate. The plate was washed and incubated for 1 h with 100 μL/well of 1:1000 dilution of HRP-conjugated anti-mouse antibody. The plate was washed and incubated with tetramethylbenzidine substrate for approximately 5 min. The reaction was stopped with equal volume of 2 M sulfuric acid, and the OD450 was measured using a plate reader.

### Example 2. An mRNA Display Selection Against K-Ras(G12V)-GTP

A major goal of this work was to develop state-specific Ras ligands. In order to enhance the targeting of the active state of Ras, our fibronectin library was constructed with a mutagenized sequence (CDRH1) derived from the known K-Ras binder, iDab#6 (Tanaka et al., Tumour prevention by a single antibody domain targeting the interaction of signal transduction proteins with RAS, EMBO J. 26 (2007) 3250-3259.) in the BC loop and a totally random FG loop (FIG. 1a). The initial diversity of the library was ˜10¹² independent sequences (˜5 copies each). mRNA display selection was performed against human K-Ras(G12V) that had been exchanged with GTPγS to create bias for binders to the active state of the target (FIG. 1b). Target specific binding was first detected in round 4 (FIG. 1b). To increase state-selective binding for active Ras, a 15-fold molar excess of K-Ras(G12V)-GDP was added in solution that would compete and remove molecules that were not specific for active Ras during the affinity enrichment step in rounds 5 and 6. The resulting pool 6 has 23% binding to K-Ras(G12V)-GTP, 16% binding to K-Ras(G12V)-GDP, and no detectable binding to the selection matrix (neutravidin-agarose or streptavidin-agarose beads; FIG. 1b). Thus, pool 6 shows a measurable preference for the GTP-bound form of K-Ras(G12V). Pool 6 was then sequenced using high-throughput sequencing techniques and ranked the selected sequences by their abundance to determine the sequences that were enriched during selection.

### Example 3. Isolation of RasIn1 Via a Cell-Based Screen

Ten high abundance sequences were tested for function using a cell-based screen we had previously developed to monitor in vivo protein binding and co-localization (Gross et al., Recombinant probes for visualizing endogenous synaptic proteins in living neurons, Neuron 78 (2013) 971-985). In this assay, the target protein is localized to the Golgi as a fusion protein bearing (1) a Golgi-targeting sequence (GTS), (2) streptavidin (SA), and (3) the target [here, H-Ras(G12V)]. We chose H-Ras for this screen to find those molecules that were Ras specific and would be able to bind to both K- and H-Ras. We also utilized the constitutively active H-Ras(G12V) mutant in order to find Ras and state-specific binders that would not be downregulated by endogenous RasGAP that might be present in the COS cells. Each candidate was screened as an eGFP fusion that was co-transfected with the target. Candidates were scored for co-localization with the target by immunocytochemistry. The best-performing candidate targeting H-Ras(G12V) was termed RasIn1 (FIG. 1c and FIG. 1d). Images of the COS cells demonstrate that eGFP-labeled RasIn1 and Golgi-targeted SA-H-Ras(G12V) (visualized with rhodamine-biotin) accurately colocalize with little excess staining elsewhere in the cell. This result indicates that RasIn1 (1) expresses stably and functionally in mammalian cells (COS cells) and (2) has low background and little, nonspecific localization in the rest of the cell. Importantly, this low background binding demonstrates that RasIn1 accurately recognizes H-Ras in the complex environment inside the cytosol.

RasIn1 is selective for the GTP-bound state, competes with Raf-RBD, and is sensitive to mutation of Switch I.

### Example 4. RasIn1 is Selective for the GTP-Bound State, Competes with Raf-RBD, and is Sensitive to Mutation of Switch I

RasIn1 shows efficient pull-down with the active (GTP-bound) form of K-Ras and less pull-down with K-Ras-GDP or beads that lack target (FIG. 2a). To determine where RasIn1 binds K-Ras, we performed competition binding experiments with a natural Ras binding partner, the Raf-RBD. Co-incubation of RasIn1 with a molar excess of Raf-RBD decreases the binding 12-fold (FIG. 2a), consistent with the two proteins competing for the same binding interface on Ras.

Co-crystal structures of H-Ras with Raf-RBD (PDBID: 3KUD) make it clear that Raf-RBD makes direct contacts with the Switch I region of Ras, proximal to the nucleotide-binding pocket. The Y32R mutation in the Switch I region of Ras has previously been shown to decrease the binding between Ras and Raf-RBD. This mutation also decreases the binding of Ras with Rasin1 (FIG. 2b). Taken together, our data argue that RasIn1 binds Ras in a state-specific fashion via recognition of the Switch I region of Ras in a functionally similar fashion to Raf-RBD.

### Example 5. RasIn1 is Specific for K- and H-Ras and Discriminates Between Highly Homologous Members of the Ras Superfamily

K- and H-Ras are members of the Ras subfamily with high sequence homology and identical Switch I sequences. To further characterize the specificity of RasIn1, we tested RasIn1 binding to wild-type K-Ras, K-Ras(G12V), wild-type H-Ras, and H-Ras(G12V) (FIG. 3a). We observed mutant-selective binding for both K- and H-Ras (GTP). We also observe similar binding to both H- and K-Ras(G12V) and to wild-type H- and K-Ras, indicating that RasIn1 is not selective for a particular isoform of Ras.

The Ras superfamily is composed of five subfamilies with related sequence and structure. For example, Ras family members include, but are not limited to, HRAS; KRAS; NRAS; KRAS 4A; KRAS 4B; DIRAS1; DIRAS2; DIRAS3; ERAS; GEM; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; and RRAS2. (Wennerberg et al., (2005) The Ras superfamily at a glance. J. Cell. Sci. 118 (5): 843-6). To demonstrate that RasIn1 specifically recognizes Ras, RasIn1 was tested against two other members of the Ras superfamily using a radioactive in vitro pull-down assay (FIG. 3b). RasIn1 shows little to no binding to Rap1B(G12V)-GTP and Arf1-GTP. The ability to discriminate between Ras and Rap1B(G12V) bears comment, as these proteins are 57% sequence identical and the Ras Switch I region (SEQ ID NO: 16 YPDTIED) differs by 2 amino acids from the Rap1B Switch I region (SEQ ID NO: 22 YDPTIED). The Ras Switch I sequence is also similar to the Switch I sequence of Arf1 (TIPTIGF) (SEQ ID NO: 23). RasIn1 is thus mutant specific (G12V versus wild-type) and state specific (GTP versus GDP) and differentiates between close Ras homologs.

### Example 6. RasIn1 Colocalizes with Active Ras In Vivo

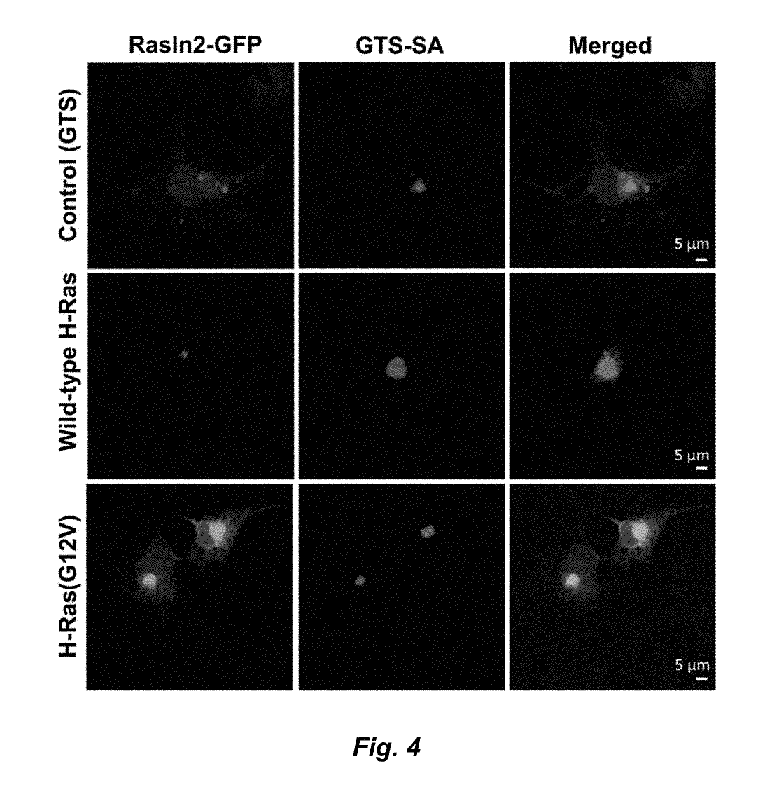

Our in vitro data indicate that RasIn1 binds to both wild-type and mutant activated Ras proteins (FIG. 3a). Using the COS cell assay (FIG. 1c), RasIn1 was tested to determine if it could also recognize wild-type H-Ras and H-Ras(G12V) inside the cells (FIG. 4). Co-localization of RasIn1 is not seen when Golgi-bound target is not expressed (FIG. 4a-c).

While RasIn1 binds to both H-Ras-GTP and H-Ras(G12V)-GTP in vitro (FIG. 3a), co-localization was observed only in the COS cell assay with H-Ras(G12V). In the cell-based assay, wild-type H-Ras shows poor binding and co-localization, while the H-Ras(G12V) mutant shows good co-localization and binding (FIG. 4). One possible explanation for this observation is that RasIn1 has higher binding for H-Ras(G12V) than wild-type H-Ras. Alternatively, wild-type H-Ras may exist in the cell in a predominantly inactive, GDP-bound state due to downregulation by endogenous Ras-GAP. This hypothesis is consistent with the wild-type protein being a substrate for hydrolysis via endogenous Ras-GAP, whereas the mutant is not.

### Example 6. Mutational Analysis of RasIn1

Mutants of the RasIn1 loops were constructed to determine functionally important residues (FIG. 5a). In the BC loop, mutation of Ser-21 to Ala (S21A) results in little decrease in pull-down efficiency, suggesting that this residue is not critical for the function of the intrabody. On the other hand, the S24A mutation results in a significant decrease in binding, while the S24R mutation that increases steric bulk at this position results in complete loss of binding. These data suggest a specific role for serine at this position. In the FG loop, the R72A and R77A mutations also abolish binding completely, suggesting that these two residues are critical for the function of RasIn1, Likewise, R80A also shows a significant decrease in binding.

High-throughput sequencing also affords new insights into positional scanning. In the high-throughput sequencing data, both the primary clone, RasIn1, and a number of sequences differ from this clone by a single mutation (FIG. 5b). These mutations likely occurred during the course of the selection, due to inherent error rates in PCR, transcription, and reverse transcription within each mRNA display selection round. Analysis of these point mutants reveals 100% conservation at positions 22, 72, 74, 76, and 77. This point mutational analysis agrees with and complements the mutational analysis above, as both analyses identify R72 and R77 as highly important residues for binding. In addition, point mutational analysis also revealed potentially important contact sites that were not explored in the directed mutational studies, such as A22 and P74 (also see Table 1). Taken together, the positional scanning data and the high-throughput sequencing mutagenesis data argue that a significant fraction of the residues on the BC and FG loops are involved and important for binding and recognition of active Ras.

TABLE 1
The highest copy number of point mutations of Rasln1.

| | | SEQ ID | | SEQ ID | Copy |
|---|---|---|---|---|---|
| Rank | BC loop | NO: | FG loop | NO: | number |
| Wt | GPVFSAYS | 40 | FRWPMPRLVR | 41 | |
| 437 | G [symbol] VFSAYS | 42 | FRWPMPRLVR | 43 | 2285 |
| 591 | GPVFSA [symbol] S | 44 | FRWPMPRLVR | 45 | 1614 |
| 862 | GPV [symbol] SAYS | 46 | FRWPMPRLVR | 47 | 1128 |
| 871 | GPVFSAYS | 48 | FRWPMPRL [symbol] R | 49 | 1114 |
| 989 | [symbol] PVFSAYS | 50 | FRWPMPRLVR | 51 | 984 |
| 1029 | G [symbol] VFSAYS | 52 | FRWPMPRLVR | 53 | 951 |
| 1298 | GPVF [symbol] AYS | 54 | FRWPMPRLVR | 55 | 739 |
| 1316 | GPVFSAYS | 56 | FRWPMPRL [symbol] R | 57 | 725 |
| 1329 | GP [symbol] FSAYS | 58 | FRWPMPRLVR | 59 | 716 |
| 1411 | GPVFSAYS | 60 | FR [symbol] PMPRLVR | 61 | 673 |
| 1453 | GPVFSAYS | 62 | FRWPMPRL [symbol] R | 63 | 646 |
| 1501 | GPVFSAYS | 64 | FRWPMPR [symbol] VR | 65 | 620 |
| 1612 | GPVFSAYS | 66 | [symbol] RWPMPRLVR | 67 | 574 |
| 1901 | GPVFSAY [symbol] | 68 | FRWPMPRLVR | 69 | 482 |
| 2071 | GPVFSAYS | 70 | FRWP [symbol] PRLVR | 71 | 440 |
| 2087 | GPVFSAYS | 72 | [symbol] RWPMPRLVR | 73 | 435 |
| 2442 | GPVFSAYS | 74 | FRWPMPRLV [symbol] | 75 | 357 |
| 2504 | GPV [symbol] SAYS | 76 | FRWPMPRLVR | 77 | 349 |
| 2534 | GPVFSAYS | 78 | FRWP [symbol] PRLVR | 79 | 345 |
| 2566 | GPVFSAYS | 80 | FRWP [symbol] PRLVR | 81 | 341 |

### Example 7. Affinity Maturation Results in a High Affinity Binder, RasIn2

An affinity maturation selection was done to improve the affinity of RasIn1. A doped mRNA display library was constructed based on the loop sequences of RasIn1 such that there was an average of 33% wild-type amino acid at each position in the starting pool. Five rounds of affinity enrichment were performed against H-Ras(G12V) in the presence of GppNHp, which, like GTPγS, allowed us to introduce bias for the active state of Ras (FIG. 6a). Additionally, during rounds 4 and 5, the binding temperature during selection was raised to 37° C. to increase selective pressure and to select for sequences with higher affinity and stability. Pool 5 correspondingly showed a pull-down efficiency of 65%, indicating the presence of high affinity molecules in the pool.

Pool 5 of the maturation selection was analyzed by high-throughput sequencing. The four highest abundance clones (Clones 1-4) were selected for further characterization. All clones contain V19 and F20 in the BC loops and highly conserved FG loops, with the only variation in FG occurring at position 79 (V and L). The high sequence conservation suggests that all the clones bind to the same epitope of Ras as RasIn1. These four clones can be further grouped into two pairs (1 and 4 versus 2 and 3) based on the similarity of each clone's BC loop (FIG. 6c). Five out of seven positions in the BC loop are identical between the two respective clones in both groups.

All four clones were tested for binding in a radioactive pull-down assay (FIG. 6b). All clones give high levels of binding compared to the parental RasIn1 clone (>70% versus ˜5%, respectively) and also show very good selectivity for Ras-GTP versus Ras-GDP (FIG. 6). We note that RasIn1 shows lower pull-down in this experiment (˜5%; FIG. 6b) that uses a relatively small amount of target, whereas in FIG. 2, FIG. 3, FIG. 4, and FIG. 5, RasIn1 gave a much higher pull-down efficiency due to the much higher target concentration on the beads that were used in those experiments. The highest affinity sequence (Clone 3), was chosen from these experiments, termed RasIn2, and was further analyzed.

### Example 8. Characterization of RasIn2

RasIn2 maintained many of the binding characteristics of its parent clone, RasIn1. RasIn2 was matured on H-Ras(G12V) and shows a preference for H-Ras and H-Ras(G12V) over wild-type K-Ras and K-Ras(G12V) (FIG. 7a). RasIn2 is state selective and preferentially binds active Ras (FIG. 7b). RasIn2 competes with Raf-RBD (FIG. 7b) and selectively recognizes K- and H-Ras over Ras family proteins Arf1 and Rap1B (FIG. 7c). RasIn2 also has reduced binding to the Y32R Switch I mutant (FIG. 7d).

Comparison of the affinity-matured clone, RasIn2, to its parent, RasIn1, allows the identification of residues that are important for binding. Affinity maturation resulted in the conservation of 8 of the 10 residues in the FG loop, demonstrating that these residues are highly optimized for binding. These conserved residues agree with the mutational analysis from alanine scanning and from point mutational analysis from high-throughput sequencing (FIG. 5b). However, only two positions are retained in the BC loop after affinity maturation, arguing that the BC loop interactions have been optimized in the context of the new FG loop in the affinity-matured clone and potentially yielded the isoform selectivity of RasIn2 for H-Ras.

The binding affinity of the two intrabodies were determined by surface plasmon resonance (SPR; FIG. 7e). These experiments indicate that RasIn1 binds H-Ras(G12V) with a K_{D} of 2.1 μM, while RasIn2 binds with a K_{D} of 120 nM. Thus, affinity maturation provided a nearly 20-fold increase in affinity, an increase of about −1.7 kcal-mol-1, versus the parent clone. This affinity is notable because it is similar to the reported K_{D} value for the Raf-RBD: wild-type H-Ras-GTP complex (K_{D}=80 nM). Furthermore, the fact that Raf-RBD binds H-Ras(G12V) with lower affinity than wild-type H-Ras indicates that RasIn2 may have a similar or greater affinity for mutant Ras compared to Raf-RBD.

An ELISA-based activity assay was used to directly compare the affinities of Raf-RBD and RasIn2 to mutant Ras (FIG. 8). In this assay, biotin-labeled H-Ras(G12V) was immobilized on a streptavidin-coated ELISA plate and then incubated with either FLAG-tagged RasIn2 or FLAG-tagged Raf-RBD. After the detection of the FLAG tag with anti-FLAG antibody, followed by a secondary anti-mouse antibody conjugated to horseradish peroxidase (HRP), RasIn2 gave ˜10-fold higher signal as compared to RBD. These data indicate that RasIn2 has a higher affinity to active H-Ras(G12V) than Raf-RBD.

Lastly, RasIn2 was tested in the cell-based co-localization assay against H-Ras(G12V) (FIG. 9) to demonstrate its specific binding inside the cells. Similar to RasIn1, RasIn2 colocalizes well with H-Ras(G12V), arguing that the protein is stable and functional in mammalian cells and recognizes activated Ras in that context. However, unlike RasIn1, RasIn2 also co-localized with wild-type H-Ras inside the cells (FIG. 9d-f). This co-localization could be due to competition between RasIn2 and RasGAP (due to the high affinity of RasIn2), thus preserving or trapping substantial amounts of H-Ras in the GTP-bound state. Alternatively, some co-localization could be from the binding of RasIn2 to the GDP-bound state of H-Ras, possibly due to the high expression levels of transfected RasIn2 and H-Ras present in the cell. Co-localization with active Ras can be observed with RasIn1 inside the cells even though the KD is ˜2.1 uM (FIG. 4), and in vitro RasIn2 has similar affinity for the GDP form of Ras as RasIn1 has for active H-Ras(G12V)-GTP (FIG. 6b).

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

AMINO ACID SEQUENCES

SEQ ID NO: 1
MLEVKEASPTSIQISWGPVFSAYSRYYRITYGETGGNSPVQEFTVPGSKS
AATISGLKPGVDYTITVYAVTFRWPMPRLVRPISINYRT
SEQ ID NO: 2
MLEVKEASPTSIQISWSIVFGKHDRYYRITYGETGGNSPVQEFTVPGSKS
AATISGLKPGVDYTITVYAVTFRWPKRRLVRPISINYRT
SEQ ID NO: 3
MLEVKEASPTSIQISWGRVFSLDSRYYRITYGETGGNSPVQEFTVPGSKS
AATISGLKPGVDYTITVYAVTFRWPNPRLVRPISINYRT
SEQ ID NO: 4
MLEVKEASPTSIQISWEYVFGRHDRYYRITYGETGGNSPVQEFTVPGSKS
AATISGLKPGVDYTITVYAVTFRWPKRRLLWPISINYRT
SEQ ID NO: 5
MLEVKEASPTSIQISWGSVFRADSRYYRITYGETGGNSPVQEFTVPGSKS
AATISGLKPGVDYTITVYAVTFRWPRPRLLWPISINYRT
SEQ ID NO: 6 GPVFSAYS
SEQ ID NO: 7 SIVFGKHD
SEQ ID NO: 8 GRVFSLDS
SEQ ID NO: 9 EYVFGRHD
SEQ ID NO: 10 GSVFRADS
SEQ ID NO: 11 FRWPMPRLVR
SEQ ID NO: 12 FRWPKRRLVR
SEQ ID NO: 13 FRWPNPRLVR
SEQ ID NO: 14 FRWPKRRLLW
SEQ ID NO: 15 FRWPRPRLLW
SEQ ID NO: 16 YPDTIED
SEQ ID NO: 17
MLEVKEASPTSIQISWXXXXXXXRYYRITYGETGGNSPVQEFTVPGSKSA
ATISGLKPGVDYTITVYAVTXXXXXXXXXXPISINYRT
SEQ ID NO: 18
VSDVPRDLEVVAATPTSLLISWDAPAVTVRYYRITYGETGGNSPVQEFTV
PGSKSTATISGLKPGVDYTITVYAVTGRGDSPASSKPISINYRT
SEQ ID NO: 19
LEVKEASPTSIQISWDAPAVTVRYYRITYGETGGNSPVQEFTVPGSKSAA
TISGLKPGVDYTITVYAVTGRGDSPASSKPISINYRT
SEQ ID NO: 20
MREYKLVVLGSGGVGKSALTVQFVQGIFVEKYDPTIEDSYRKQVEVDAQQ
CMLEILDTAGTEQFTAMRDLYMKNGQGFALVYSITAQSTFNDLQDLREQI
LRVKDTDDVPMILVGNKCDLEDERVVGKEQGQNLARQWNNCAFLESSAKS
KINVNEIFYDLVRQINRKTPVPGKARKKSSCQLL
SEQ ID NO: 21
MGNIFANLFKGLFGKKEMRILMVGLDAAGKTTILYKLKLGEIVTTIPTIG
FNVETVEYKNISFTVWDVGGQDKIRPLWRHYFQNTQGLIFVVDSNDRERV
NEAREELMRMLAEDELRDAVLLVFANKQDLPNAMNAAEITDKLGLHSLRH
RNWYIQATCATSGDGLYEGLDWLSNQLRNQK
SEQ ID NO: 22 YDPTIED
SEQ ID NO: 23 TIPTIGF
SEQ ID NO: 24
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM
SEQ ID NO: 25
MTEYKLVVVGAGGVGKSALTIQLIQNHFVDEYDPTEDSYRKQVVIDGETC
LLDILDTAGQEEYSAMRDQYMRTGEGFLCVFAINNTKSFEDIHQYREQIK
RVKDSDDVPMVLVGNKCDLAARTVESRQAQDLARSYGIPYIETSAKTRQG
VEDAFYTLVREIRQHKLRKLNPPDESGPGCMSCKCVLS
SEQ ID NO: 26
MTEYKLVVVGAVGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM
SEQ ID NO: 27
MTEYKLVVVGAVGVGKSALTIQLIQNHFVDEYDPTIEDSYRKQVVIDGET
CLLDILDTAGQEEYSAMRDQYMRTGEGFLCVFAINNTKSFEDIHQYREQI
KRVKDSDDVPMVLVGNKCDLAARTVESRQAQDLARSYGIPYIETSAKTRQ
GVEDAFYTLVREIRQHKLRKLNPPDESGPGCMSCKCVLS
SEQ ID NO: 28
MTEYKLVVVGAVGVGKSALTIQLIQNHFVDERDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM
SEQ ID NO: 29
MTEYKLVVVGAVGVGKSALTIQLIQNHFVDERDPTIEDSYRKQVVIDGET
CLLDILDTAGQEEYSAMRDQYMRTGEGFLCVFAINNTKSFEDIHQYREQI
KRVKDSDDVPMVLVGNKCDLAARTVESRQAQDLARSYGIPYIETSAKTRQ
GVEDAFYTLVREIRQHKLRKLNPPDESGPGCMSCKCVLS
SEQ ID NO: 30
MREYKLVVLGSVGVGKSALTVQFVQGIFVEKYDPTIEDSYRKQVEVDAQQ
CMLEILDTAGTEQFTAMRDLYMKNGQGFALVYSITAQSTFNDLQDLREQI
LRVKDTDDVPMILVGNKCDLEDERVVGKEQGQNLARQWNNCAFLESSAKS
KINVNEIFYDLVRQINRKTPVPGKARKKSSCQLL
SEQ ID NO: 31
MREYKLVVLGSVGVGKSALTVQFVQGIFVEKRDPTIEDSYRKQVEVDAQQ
CMLEILDTAGTEQFTAMRDLYMKNGQGFALVYSITAQSTFNDLQDLREQI
LRVKDTDDVPMILVGNKCDLEDERVVGKEQGQNLARQWNNCAFLESSAKS
KINVNEIFYDLVRQINRKTPVPGKARKKSSCQLL

What is claimed is:

1. An isolated polypeptide comprising a fibronectin domain, and a first peptide domain at least 90% identical to an amino acid sequence selected from a group consisting of SEQ ID NO: 6, SEQ ID NO: 7, SEQ ID NO: 8, SEQ ID NO: 9, and SEQ ID NO: 10, and a second peptide domain at least 90% identical to an amino acid sequence selected from a group consisting of SEQ ID NO: 11, SEQ ID NO: 12, SEQ ID NO: 13, SEQ ID NO: 14, and SEQ ID NO: 15, that specifically binds to an activated Ras protein.

2. The polypeptide of claim 1 wherein the first peptide domain is SEQ ID NO: 6 and the second peptide domain is SEQ ID NO: 11.

3. The polypeptide of claim 1 wherein the first peptide domain is SEQ ID NO: 7 and the second peptide domain is SEQ ID NO: 12.

4. The polypeptide of claim 1 wherein the fibronectin domain comprises a 10^{th} domain of fibronectin.

5. The polypeptide of claim 4 wherein the fibronectin domain comprises 10FnIII or e10FnIII.

6. The polypeptide of claim 1 wherein a K_{D} of the polypeptide for the Ras protein is between about 5 μM and about 150 nM.

7. The polypeptide of claim 1 wherein the polypeptide is SEQ ID NO: 1 or SEQ ID NO: 2.

8. The polypeptide of claim 1 wherein the polypeptide selectively binds GTP-bound K-Ras or GTP-bound H-Ras relative to GDP-bound K-Ras, nucleotide-free K-Ras, GDP-bound H-Ras, or nucleotide-free H-Ras.

9. The polypeptide of claim 1 wherein the polypeptide selectively binds a G12V mutant K-Ras relative to wild-type K-Ras.

10. The polypeptide of claim 1 wherein the polypeptide selectively binds the Ras protein relative to a Ras family member selected from Arf1 and Rap1B.

11. The polypeptide of claim 1 wherein the polypeptide binds to SEQ ID NO: 16 of the Ras protein.

12. The polypeptide of claim 1 wherein the Ras protein comprises at least one of wild-type H-Ras, wild-type K-Ras, mutant H-Ras, or mutant K-Ras.

13. The polypeptide of claim 2 wherein the mutant H-Ras or the mutant K-Ras comprises a polypeptide sequence comprising a G12V point mutation.

14. A method of treating cancer in a subject, the method comprising administering to the subject a therapeutically effective amount of the polypeptide of claim 1.

15. A method of detecting cancer in a sample from a subject comprising contacting the sample with the polypeptide of claim 1.

16. The method of claim 15 wherein the polypeptide further comprises a detectable label.

17. The method of claim 16 wherein the detectable label is GFP or eGFP.

18. A polynucleotide encoding the polypeptide of claim 1.

19. An expression vector comprising the polynucleotide of claim 18.


SEQUENCE LISTING (82 sequences)

SEQ ID NO: 1 - protein, 89 aa, Artificial Sequence
  Description of Artificial Sequence: Synthetic polypeptide
MLEVKEASPTSIQISWGPVFSAYSRYYRITYGETGGNSPVQEFTVPGSKSAATISGLKPGVDYTITVYAVTFRWPMPRLVRPISINYRT

SEQ ID NO: 2 - protein, 89 aa, Artificial Sequence
  Description of Artificial Sequence: Synthetic polypeptide
MLEVKEASPTSIQISWSIVFGKHDRYYRITYGETGGNSPVQEFTVPGSKSAATISGLKPGVDYTITVYAVTFRWPKRRLVRPISINYRT

SEQ ID NO: 3 - protein, 89 aa, Artificial Sequence
  Description of Artificial Sequence: Synthetic polypeptide
MLEVKEASPTSIQISWGRVFSLDSRYYRITYGETGGNSPVQEFTVPGSKSAATISGLKPGVDYTITVYAVTFRWPNPRLVRPISINYRT

SEQ ID NO: 4 - protein, 89 aa, Artificial Sequence
  Description of Artificial Sequence: Synthetic polypeptide
MLEVKEASPTSIQISWEYVFGRHDRYYRITYGETGGNSPVQEFTVPGSKSAATISGLKPGVDYTITVYAVTFRWPKRRLLWPISINYRT

SEQ ID NO: 5 - protein, 89 aa, Artificial Sequence
  Description of Artificial Sequence: Synthetic polypeptide
MLEVKEASPTSIQISWGSVFRADSRYYRITYGETGGNSPVQEFTVPGSKSAATISGLKPGVDYTITVYAVTFRWPRPRLLWPISINYRT

SEQ ID NO: 6 - protein, 8 aa, Artificial Sequence
  Description of Artificial Sequence: Synthetic peptide
GPVFSAYS

SEQ ID NO: 7 - protein, 8 aa, Artificial Sequence
  Description of Artificial Sequence: Synthetic peptide
SIVFGKHD

SEQ ID NO: 8 - protein, 8 aa, Artificial Sequence
  Description of Artificial Sequence: Synthetic peptide
GRVFSLDS

SEQ ID NO: 9 - protein, 8 aa, Artificial Sequence
  Description of Artificial Sequence: Synthetic peptide
EYVFGRHD

SEQ ID NO: 10 - protein, 8 aa, Artificial Sequence
  Description of Artificial Sequence: Synthetic peptide
GSVFRADS

SEQ ID NO: 11 - protein, 10 aa, Artificial Sequence
  Description of Artificial Sequence: Synthetic peptide
FRWPMPRLVR

SEQ ID NO: 12 - protein, 10 aa, Artificial Sequence
  Description of Artificial Sequence: Synthetic peptide
FRWPKRRLVR

SEQ ID NO: 13 - protein, 10 aa, Artificial Sequence
  Description of Artificial Sequence: Synthetic peptide
FRWPNPRLVR

SEQ ID NO: 14 - protein, 10 aa, Artificial Sequence
  Description of Artificial Sequence: Synthetic peptide
FRWPKRRLLW

SEQ ID NO: 15 - protein, 10 aa, Artificial Sequence
  Description of Artificial Sequence: Synthetic peptide
FRWPRPRLLW

SEQ ID NO: 16 - protein, 7 aa, Artificial Sequence
  Description of Artificial Sequence: Synthetic peptide
YPDTIED

SEQ ID NO: 17 - protein, 88 aa, Homo sapiens
  MOD_RES (17)..(23): Any amino acid
  MOD_RES (71)..(80): Any amino acid
MLEVKEASPTSIQISWXXXXXXXRYYRITYGETGGNSPVQEFTVPGSKSAATISGLKPGVDYTITVYAVTXXXXXXXXXXPISINYRT

SEQ ID NO: 18 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 19 - protein, 87 aa, Homo sapiens
LEVKEASPTSIQISWDAPAVTVRYYRITYGETGGNSPVQEFTVPGSKSAATISGLKPGVDYTITVYAVTGRGDSPASSKPISINYRT

SEQ ID NO: 20 - protein, 184 aa, Artificial Sequence
  Description of Artificial Sequence: Synthetic polypeptide
MREYKLVVLGSGGVGKSALTVQFVQGIFVEKYDPTIEDSYRKQVEVDAQQCMLEILDTAGTEQFTAMRDLYMKNGQGFALVYSITAQSTFNDLQDLREQILRVKDTDDVPMILVGNKCDLEDERVVGKEQGQNLARQWNNCAFLESSAKSKINVNEIFYDLVRQINRKTPVPGKARKKSSCQLL

SEQ ID NO: 21 - protein, 181 aa, Artificial Sequence
  Description of Artificial Sequence: Synthetic polypeptide
MGNIFANLFKGLFGKKEMRILMVGLDAAGKTTILYKLKLGEIVTTIPTIGFNVETVEYKNISFTVWDVGGQDKIRPLWRHYFQNTQGLIFVVDSNDRERVNEAREELMRMLAEDELRDAVLLVFANKQDLPNAMNAAEITDKLGLHSLRHRNWYIQATCATSGDGLYEGLDWLSNQLRNQK

SEQ ID NO: 22 - protein, 7 aa, Artificial Sequence
  Description of Artificial Sequence: Synthetic peptide
YDPTIED

SEQ ID NO: 23 - protein, 7 aa, Artificial Sequence
  Description of Artificial Sequence: Synthetic peptide
TIPTIGF

SEQ ID NO: 24 - protein, 188 aa, Homo sapiens [sequence omitted]

SEQ ID NO: 25 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 26 - protein, 188 aa, Artificial Sequence
  Description of Artificial Sequence: Synthetic polypeptide
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 27 - protein, 189 aa, Artificial Sequence [sequence omitted]

SEQ ID NO: 28 - protein, 188 aa, Artificial Sequence
  Description of Artificial Sequence: Synthetic polypeptide
MTEYKLVVVGAVGVGKSALTIQLIQNHFVDERDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 29 - protein, 189 aa, Artificial Sequence
  Description of Artificial Sequence: Synthetic polypeptide
MTEYKLVVVGAVGVGKSALTIQLIQNHFVDERDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 30 - protein, 184 aa, Artificial Sequence
  Description of Artificial Sequence: Synthetic polypeptide
MREYKLVVLGSVGVGKSALTVQFVQGIFVEKYDPTIEDSYRKQVEVDAQQCMLEILDTAGTEQFTAMRDLYMKNGQGFALVYSITAQSTFNDLQDLREQILRVKDTDDVPMILVGNKCDLEDERVVGKEQGQNLARQWNNCAFLESSAKSKINVNEIFYDLVRQINRKTPVPGKARKKSSCQLL

SEQ ID NO: 31 - protein, 184 aa, Artificial Sequence
  Description of Artificial Sequence: Synthetic polypeptide
MREYKLVVLGSVGVGKSALTVQFVQGIFVEKRDPTIEDSYRKQVEVDAQQCMLEILDTAGTEQFTAMRDLYMKNGQGFALVYSITAQSTFNDLQDLREQILRVKDTDDVPMILVGNKCDLEDERVVGKEQGQNLARQWNNCAFLESSAKSKINVNEIFYDLVRQINRKTPVPGKARKKSSCQLL

SEQ ID NO: 32-39 - skipped (8 entries)

SEQ ID NO: 40 - protein, 8 aa, Homo sapiens
GPVFSAYS

SEQ ID NO: 41 - protein, 10 aa, Homo sapiens
FRWPMPRLVR

SEQ ID NO: 42 - protein, 8 aa, Artificial Sequence
  Description of Artificial Sequence: Synthetic peptide
GSVFSAYS

SEQ ID NO: 43 - protein, 10 aa, Artificial Sequence
  Description of Artificial Sequence: Synthetic peptide
FRWPMPRLVR

SEQ ID NO: 44 - protein, 8 aa, Artificial Sequence
  Description of Artificial Sequence: Synthetic peptide
GPVFSASS

SEQ ID NO: 45 - protein, 10 aa, Artificial Sequence
  Description of Artificial Sequence: Synthetic peptide
FRWPMPRLVR

SEQ ID NO: 46 - protein, 8 aa, Artificial Sequence
  Description of Artificial Sequence: Synthetic peptide
GPVLSAYS

SEQ ID NO: 47 - protein, 10 aa, Artificial Sequence
  Description of Artificial Sequence: Synthetic peptide
FRWPMPRLVR

SEQ ID NO: 48 - protein, 8 aa, Artificial Sequence
  Description of Artificial Sequence: Synthetic peptide
GPVFSAYS

SEQ ID NO: 49 - protein, 10 aa, Artificial Sequence
  Description of Artificial Sequence: Synthetic peptide
FRWPMPRLGR

SEQ ID NO: 50 - protein, 8 aa, Artificial Sequence
  Description of Artificial Sequence: Synthetic peptide
SPVFSAYS

SEQ ID NO: 51 - protein, 10 aa, Artificial Sequence
  Description of Artificial Sequence: Synthetic peptide
FRWPMPRLVR

SEQ ID NO: 52 - protein, 8 aa, Artificial Sequence
  Description of Artificial Sequence: Synthetic peptide
GLVFSAYS

SEQ ID NO: 53 - protein, 10 aa, Artificial Sequence
  Description of Artificial Sequence: Synthetic peptide
FRWPMPRLVR

SEQ ID NO: 54 - protein, 8 aa, Artificial Sequence
  Description of Artificial Sequence: Synthetic peptide
GPVFGAYS

SEQ ID NO: 55 - protein, 10 aa, Artificial Sequence
  Description of Artificial Sequence: Synthetic peptide
FRWPMPRLVR

SEQ ID NO: 56 - protein, 8 aa, Artificial Sequence
  Description of Artificial Sequence: Synthetic peptide
GPVFSAYS

SEQ ID NO: 57 - protein, 10 aa, Artificial Sequence
  Description of Artificial Sequence: Synthetic peptide
FRWPMPRLAR

SEQ ID NO: 58 - protein, 8 aa, Artificial Sequence
  Description of Artificial Sequence: Synthetic peptide
GPAFSAYS

SEQ ID NO: 59 - protein, 10 aa, Artificial Sequence
  Description of Artificial Sequence: Synthetic peptide
FRWPMPRLVR

SEQ ID NO: 60 - protein, 8 aa, Artificial Sequence
  Description of Artificial Sequence: Synthetic peptide
GPVFSAYS

SEQ ID NO: 61 - protein, 10 aa, Artificial Sequence
  Description of Artificial Sequence: Synthetic peptide
FRRPMPRLVR

SEQ ID NO: 62 - protein, 8 aa, Artificial Sequence
  Description of Artificial Sequence: Synthetic peptide
GPVFSAYS

SEQ ID NO: 63 - protein, 10 aa, Artificial Sequence
  Description of Artificial Sequence: Synthetic peptide
FRWPMPRLMR

SEQ ID NO: 64 - protein, 8 aa, Artificial Sequence
  Description of Artificial Sequence: Synthetic peptide
GPVFSAYS

SEQ ID NO: 65 - protein, 10 aa, Artificial Sequence
  Description of Artificial Sequence: Synthetic peptide
FRWPMPRPVR

SEQ ID NO: 66 - protein, 8 aa, Artificial Sequence
  Description of Artificial Sequence: Synthetic peptide
GPVFSAYS

SEQ ID NO: 67 - protein, 10 aa, Artificial Sequence
  Description of Artificial Sequence: Synthetic peptide
LRWPMPRLVR

SEQ ID NO: 68 - protein, 8 aa, Artificial Sequence
  Description of Artificial Sequence: Synthetic peptide
GPVFSAYG

SEQ ID NO: 69 - protein, 10 aa, Artificial Sequence
  Description of Artificial Sequence: Synthetic peptide
FRWPMPRLVR

SEQ ID NO: 70 - protein, 8 aa, Artificial Sequence
  Description of Artificial Sequence: Synthetic peptide
GPVFSAYS

SEQ ID NO: 71 - protein, 10 aa, Artificial Sequence
  Description of Artificial Sequence: Synthetic peptide
FRWPTPRLVR

SEQ ID NO: 72 - protein, 8 aa, Artificial Sequence
  Description of Artificial Sequence: Synthetic peptide
GPVFSAYS

SEQ ID NO: 73 - protein, 10 aa, Artificial Sequence
  Description of Artificial Sequence: Synthetic peptide
SRWPMPRLVR

SEQ ID NO: 74 - protein, 8 aa, Artificial Sequence
  Description of Artificial Sequence: Synthetic peptide
GPVFSAYS

SEQ ID NO: 75 - protein, 10 aa, Artificial Sequence
  Description of Artificial Sequence: Synthetic peptide
FRWPMPRLVG

SEQ ID NO: 76 - protein, 8 aa, Artificial Sequence
  Description of Artificial Sequence: Synthetic peptide
GPVSSAYS

SEQ ID NO: 77 - protein, 10 aa, Artificial Sequence
  Description of Artificial Sequence: Synthetic peptide
FRWPMPRLVR

SEQ ID NO: 78 - protein, 8 aa, Artificial Sequence
  Description of Artificial Sequence: Synthetic peptide
GPVFSAYS

SEQ ID NO: 79 - protein, 10 aa, Artificial Sequence
  Description of Artificial Sequence: Synthetic peptide
FRWPVPRLVR

SEQ ID NO: 80 - protein, 8 aa, Artificial Sequence
  Description of Artificial Sequence: Synthetic peptide
GPVFSAYS

SEQ ID NO: 81 - protein, 10 aa, Artificial Sequence
  Description of Artificial Sequence: Synthetic peptide
FRWPIPRLVR

SEQ ID NO: 82 - protein, 8 aa, Artificial Sequence
  Description of Artificial Sequence: Synthetic peptide
GFTFSTFS